(19) United States
(12) Patent Application Publication
(10) Pub. No.: US 2025/0022128 A1
(43) Pub. Date: Jan. 16, 2025

(54) ARTIFICIAL INTELLIGENCE-BASED METHODS FOR GRADING, SEGMENTING, AND/OR ANALYZING LUNG ADENOCARCINOMA PATHOLOGY SLIDES

(71) Applicant: H. Lee Moffitt Cancer Center and Research Institute, Inc., Tampa, FL (US)

(72) Inventors: Elsa FLORES, Tampa, FL (US); John LOCKHART, Wesley Chapel, FL (US); Olya STRINGFIELD, Odessa, FL (US); Mahmoud ABDALAH, Wesley Chapel, FL (US)

(21) Appl. No.: 18/712,763

(22) PCT Filed: Nov. 23, 2022
(86) PCT No.: PCT/US2022/050865

§ 371 Date: May 23, 2024

Related U.S. Application Data
(60) Provisional application No. 63/282,214, filed on Nov. 23, 2021.

(51) Int. Cl. G06T 7/00; G06T 7/11

(52) U.S. Cl. CPC G06T 7/0012; G06T 7/11; G06T 2207/20081; G06T 2207/30061

(57) ABSTRACT

An example method for grading, segmenting, and analyzing lung adenocarcinoma (LUAD) pathology slides using artificial intelligence is described herein. The method includes receiving a digital pathology image of a LUAD tissue sample; inputting the digital pathology image into an artificial intelligence model; and grading, using the artificial intelligence model, the one or more tumors within the LUAD tissue sample.

### CROSS-REFERENCE TO RELATED APPLICATIONS

This application claims priority to and incorporates by reference herein U.S. Patent Application Ser. No. 63/282,214, entitled ARTIFICIAL INTELLIGENCE-BASED METHODS FOR GRADING, SEGMENTING, AND/OR ANALYZING LUNG ADENOCARCINOMA PATHOLOGY SLIDES, the contents of which is hereby incorporated by this reference in its entirety as if fully set forth herein.

### STATEMENT REGARDING FEDERALLY FUNDED RESEARCH

This invention was made with government support under Grant nos. R35CA197452 and T32CA233399-03 awarded by the National Institutes of Health/National Cancer Institute. The government has certain rights in the invention.

### SUMMARY

An example method for grading, segmenting, and analyzing lung adenocarcinoma (LUAD) pathology slides using artificial intelligence is described herein. The method includes receiving a digital pathology image of a LUAD tissue sample; inputting the digital pathology image into an artificial intelligence model; and grading, using the artificial intelligence model, the one or more tumors within the LUAD tissue sample.

The step of grading optionally includes assigning each of the one or more tumors to one of a plurality of classes. For example, the classes can include one or more of normal alveolar, normal bronchiolar, Grade 1 LUAD, Grade 2 LUAD, Grade 3 LUAD, Grade 4 LUAD, and Grade 5 LUAD. Alternatively or additionally, the step of grading includes generating graphical display data for a pseudo color map of the one or more tumors.

In some implementations, the step of grading, using the artificial intelligence model, the one or more tumors comprises assigning one or more areas within each of the one or more tumors to one of a plurality of classes on a pixel-by-pixel basis or a cell-by-cell basis.

In some implementation, the method further comprises, identifying, based at least on the pixel-by-pixel or cell-by-cell assignments, one or more genes of interest or one or more drivers of tumor progression.

In some implementations, the method further includes segmenting, using the artificial intelligence model, the one or more tumors in the digital pathology image.

In some implementations, the method further includes analyzing the one or more tumors. The step of analyzing optionally includes counting the one or more tumors. Alternatively or additionally, the step of analyzing optionally includes characterizing an intratumor heterogeneity of the one or more tumors.

In some implementations, the method further includes performing an immuno-histochemistry (IHC) analysis of the one or more tumors.

In some implementations, the artificial intelligence model is a machine learning model. For example, the machine learning model can optionally be a supervised machine learning model such as a convolutional neural network (CNN).

In some implementations, the example supervised machine learning model comprises one or more Residual Neural Network (ResNet) layers or components.

In some implementations, the supervised machine learning model further comprises one or more atrous convolutional layers and/or one or more transposed convolutional layers.

In some implementations, the digital pathology image is a hematoxylin & eosin (H&E) stained slide image. Optionally, the LUAD tissue sample is from a mouse. Alternatively, the LUAD tissue sample is optionally from a human.

An example method for integrating an immuno-histochemistry (IHC analysis) with an artificial intelligence-based LUAD tissue sample analysis is described herein. The method includes receiving a first digital pathology image of a first LUAD tissue sample, the first digital pathology image being a hematoxylin & eosin (H&E) stained slide image; inputting the first digital pathology image into an artificial intelligence model; grading, using the artificial intelligence model, one or more tumors within the first LUAD tissue sample; and segmenting, using the artificial intelligence model, the one or more tumors in the digital pathology image. Additionally, the method includes receiving a second digital pathology image comprising a second LUAD tissue sample, the second digital pathology image being an immuno-stained slide image; and identifying and classifying a plurality of positively and negatively stained cells within the second LUAD tissue sample. The method further includes co-registering the first and second digital pathology images; and projecting a plurality of respective coordinates of the positively and negatively stained cells within the second LUAD tissue sample onto the one or more tumors within the first LUAD tissue sample.

An example transfer learning method is also described herein. The method includes training a machine learning model with a dataset, where the dataset includes a plurality of mouse model digital pathology images. Each of the mouse model digital pathology images is of a respective lung LUAD tissue sample from a mouse. The method further includes receiving a digital pathology image of a LUAD tissue sample from a human; inputting the digital pathology image into the trained machine learning model; and grading, using the trained machine learning model, one or more tumors within the LUAD tissue sample from the human.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is an example computing device.

FIG. 2A depicts representations of training data network architecture in accordance with certain embodiments of the present disclosure.

FIG. 2B depicts representations of training data multiple output types in accordance with certain embodiments of the present disclosure.

FIG. 2C depicts representations of training data output scaling in accordance with certain embodiments of the present disclosure.

FIG. 2D depicts representations of training data employed and produced by Grading of Lung Adenocarcinoma with Simultaneous Segmentation by Artificial Intelligence (GLASS-AI) in accordance with certain embodiments of the present disclosure.

FIG. 3A depicts examples of hematoxylin and eosin-stained whole slide images of mouse models of lung adenocarcinoma in accordance with certain embodiments of the present disclosure.

FIG. 3B depicts examples of manual annotation of tumors and normal regions by a human rater in accordance with certain embodiments of the present disclosure.

FIG. 4A depicts examples of manual annotation of tumors and normal regions by a human rater in accordance with certain embodiments of the present disclosure.

FIG. 4B depicts examples of tumor grading maps produced by GLASS-AI in accordance with certain embodiments of the present disclosure.

FIG. 5A depicts examples of manual annotation of tumors and normal regions by a human rater in which arrows indicate tumors example tumors that had overall tumor grades that were determined by a small amount of higher-grade tumor in accordance with certain embodiments of the present disclosure.

FIG. 5B depicts example maps of overall tumor grades produced by GLASS-AI in which arrows indicate tumors example tumors that had overall tumor grades that were determined by a small amount of higher-grade tumor in accordance with certain embodiments of the present disclosure.

FIG. 6A depicts examples of manual annotation of tumors and normal regions by a human rater in accordance with certain embodiments of the present disclosure.

FIG. 6B depicts example maps of overall tumor grades produced by GLASS-AI in accordance with certain embodiments of the present disclosure.

FIG. 7A is a graph depicting F1 scores in accordance with certain embodiments of the present disclosure.

FIG. 7B is a graph depicting total annotation areas in accordance with certain embodiments of the present disclosure.

FIG. 7C is a schematic representation depicting a multi-class confusion matrix in accordance with certain embodiments of the present disclosure.

FIG. 7D is an example depicting tumor grading and segmentation by GLASS-AI compared to a human rater on a set of whole slide images of mouse models of lung adenocarcinoma and examples of tumor regions identified exclusively by GLASS-AI in accordance with certain embodiments of the present disclosure.

FIG. 7E is an example depicting tumor grading and segmentation exclusively by a human rater in accordance with certain embodiments of the present disclosure.

FIG. 7F is an example depicting highly heterogenous tumors in accordance with certain embodiments of the present disclosure.

FIG. 7G is another example depicting highly heterogenous tumors in accordance with certain embodiments of the present disclosure.

FIG. 8 is an example of the classification limitation of GLASS-AI on unlearned tissue features such as blood vessels in accordance with certain embodiments of the present disclosure.

FIG. 9A is an example of manual analysis of genetically engineered mouse models of lung adenocarcinoma in accordance with certain embodiments of the present disclosure.

FIG. 9B is an example of manual analysis of genetically engineered mouse models to uncover differences in tumor counts in accordance with certain embodiments of the present disclosure.

FIG. 9C is an example of manual analysis of genetically engineered mouse models to uncover differences in tumor burden in accordance with certain embodiments of the present disclosure.

FIG. 10 is a table of the grading agreement between GLASS-AI and a human rater on a set of 10 whole slide images of mouse models of lung adenocarcinoma in accordance with certain embodiments of the present disclosure.

FIG. 11A is an example of applying GLASS-AI to analyze genetically engineered mouse models of lung adenocarcinoma to quantify differences in tumor counts in accordance with certain embodiments of the present disclosure.

FIG. 11B is an example of applying GLASS-AI to analyze genetically engineered mouse models of lung adenocarcinoma to quantify differences in tumor burden in accordance with certain embodiments of the present disclosure.

FIG. 11C is an example of applying GLASS-AI to analyze genetically engineered mouse models of lung adenocarcinoma to quantify differences in tumor size distribution in accordance with certain embodiments of the present disclosure.

FIG. 11D is an example of applying GLASS-AI to quantify differences in average tumor sizes between different genetically engineered mouse models of lung adenocarcinoma in accordance with certain embodiments of the present disclosure.

FIG. 11E is an example of applying GLASS-AI to quantify differences in the distribution of tumor sizes between different genetically engineered mouse models of lung adenocarcinoma in accordance with certain embodiments of the present disclosure.

FIG. 11F is an example of the size of tumor of various tumor grades in accordance with certain embodiments of the present disclosure.

FIG. 11G is an example of applying GLASS-AI to quantify differences in the distribution of tumor sizes across various tumor grades between different genetically engineered mouse models of lung adenocarcinoma in accordance with certain embodiments of the present disclosure.

FIG. 12A is an example of applying GLASS-AI to analyze the intratumor heterogeneity of lung adenocarcinoma in genetically engineered mouse models that is obfuscated by overall tumor grading by a human rater in accordance with certain embodiments of the present disclosure.

FIG. 12B is an example of applying GLASS-AI to analyze the intratumor heterogeneity of lung adenocarcinoma in genetically engineered mouse models that is contained within tumors of different grades in accordance with certain embodiments of the present disclosure.

FIG. 12C is an example of applying GLASS-AI to analyze the intratumor heterogeneity of lung adenocarcinoma in genetically engineered mouse models that varies between experimental genotypes in accordance with certain embodiments of the present disclosure.

FIG. 12D is an example of applying GLASS-AI to analyze the intratumor heterogeneity of lung adenocarcinoma in genetically engineered mouse models among tumor grades in accordance with certain embodiments of the present disclosure.

FIG. 13 is a representative diagram of human lung adenocarcinoma tissue microarray core cross-sections with clinical grading and analysis by GLASS-AI in accordance with certain embodiments of the present disclosure.

FIG. 14A is an example of applying GLASS-AI to analyze human lung adenocarcinoma tissue microarray core cross-sections in comparison to clinical analysis to identify tumor and normal cores in accordance with certain embodiments of the present disclosure.

FIG. 14B is an example of correspondence between GLASS-AI and clinical grading using two algorithms to assign overall tumor grades based on the output of GLASS-AI in accordance with certain embodiments of the present disclosure.

FIG. 15A is an example of analyzing progression-free survival based on clinical grading in accordance with certain embodiments of the present disclosure.

FIG. 15B is an example of applying GLASS-AI to stratify patients using two algorithms to assign overall tumor grades based on the output of GLASS-AI in accordance with certain embodiments of the present disclosure.

FIG. 16A is an example of applying GLASS-AI to further stratify patients within the well-differentiated clinical grades in accordance with certain embodiments of the present disclosure.

FIG. 16B is an example of applying GLASS-AI to further stratify patients within the moderately differentiated clinical grades in accordance with certain embodiments of the present disclosure.

FIG. 16C is an example of applying GLASS-AI to further stratify patients within the poorly differentiated clinical grades in accordance with certain embodiments of the present disclosure.

FIG. 17 is a diagram of the integration of tumor grading of lung adenocarcinoma by GLASS-AI and immunohistochemical staining of tissue sections adjacent to the hematoxylin and eosin-stained section used for tumor grading.

FIG. 18A is an example of applying the integration of GLASS-AI and immunohistochemical staining of adjacent tissue sections in accordance with certain embodiments of the present disclosure.

FIG. 18B is an example of applying the integration of GLASS-AI and immunohistochemical staining to examine associations between staining positivity and overall tumor grade in accordance with certain embodiments of the present disclosure.

FIG. 19A is an example of applying the integration of GLASS-AI and immunohistochemical staining of adjacent tissue sections in accordance with certain embodiments of the present disclosure.

FIG. 19B is an example of applying the integration of GLASS-AI and immunohistochemical staining to examine associations between staining positivity and distances from tumor edges in accordance with certain embodiments of the present disclosure.

FIG. 20 is a schematic diagram of the workflow utilized to integrate the tumor grading output of GLASS-AI with immunohistochemical stain analysis on adjacent tissue sections in accordance with certain embodiments of the present disclosure.

FIG. 21 is a schematic diagram of modified alleles employed in genetically engineered mouse models of lung adenocarcinoma in accordance with certain embodiments of the present disclosure.

FIG. 22A is an example of applying the integration of GLASS-AI and immunohistochemical staining to examine the alteration of staining positivity in tumors in accordance with certain embodiments of the present disclosure.

FIG. 22B is an example of applying the integration of GLASS-AI and immunohistochemical staining using adjacent tissue sections in accordance with certain embodiments of the present disclosure.

FIG. 22C is an example of applying the integration of GLASS-AI and immunohistochemical staining and staining distribution in tumors in accordance with certain embodiments of the present disclosure.

FIG. 22D is an example of applying the integration of GLASS-AI and immunohistochemical staining and staining distribution in tumors in accordance with certain embodiments of the present disclosure.

FIG. 22E is an example of applying the integration of GLASS-AI and immunohistochemical staining and regions of various grades within individual tumors in accordance with certain embodiments of the present disclosure.

FIG. 23 is an example of a graphical user interface used to input images for analysis by GLASS-AI, define analysis parameters, and adjust output characteristics in accordance with certain embodiments of the present disclosure.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” an, “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The term “tumor” is defined herein as an abnormal mass of hyperproliferative or neoplastic cells from a tissue other than blood, bone marrow, or the lymphatic system, which may be benign or cancerous. In general, the tumors described herein are cancerous. As used herein, the terms “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of solid cancerous growths, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. Examples of solid tumors are sarcomas, carcinomas, and lymphomas. Leukemias (cancers of the blood) generally do not form solid tumors.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Examples include, but are not limited to, lung carcinoma, adrenal carcinoma, rectal carcinoma, colon carcinoma, esophageal carcinoma, prostate carcinoma, pancreatic carcinoma, head and neck carcinoma, or melanoma. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, classification, etc. from raw data. Representation learning techniques include, but are not limited to, autoencoders. The term “deep learning” is defined herein to be a subset of machine learning that that enables a machine to automatically discover representations needed for feature detection, prediction, classification, etc. in raw data using layers of processing. Deep learning techniques include, but are not limited to, artificial neural network or multilayer perceptron (MLP).

Machine learning techniques include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

### Example Artificial Intelligence-Based Methods for Studying Lung Adenocarcinoma Pathology Slides

In one example described herein, a method for grading, segmenting, and analyzing lung adenocarcinoma (LUAD) pathology slides using artificial intelligence is provided. The method includes receiving a digital pathology image of a LUAD tissue sample. The digital pathology image can be a whole slide image (WSI) or an image field captured from a microscope. For example, in some implementations, the digital pathology image is a hematoxylin & eosin (H&E) stained slide image. Optionally, in some implementations, the LUAD tissue sample is from a mouse. Alternatively, in other implementations, the LUAD tissue sample is optionally from a human.

The method also includes inputting the digital pathology image into an artificial intelligence model. Additionally, as described herein, the digital pathology image is optionally divided into patches/tiles before being input into the artificial intelligence model. It should be understood that such artificial intelligence model is operating in inference mode. In other words, such artificial intelligence model was previously trained with a data set (or dataset) to map an input (also referred to as feature or features) to an output (also referred to as target or targets). In some implementations, the artificial intelligence model is a machine learning model. For example, the machine learning model can optionally be a supervised machine learning model such as a convolutional neural network (CNN), multilayer perceptron, or support-vector machine. An example CNN architecture is described herein.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a data set to maximize or minimize an objective function. In some implementations, the objective function is a cost function, which is a measure of the ANN's performance (e.g., error such as L1 or L2 loss) during training. The training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation. It should be understood that an artificial neural network is provided only as an example machine learning model. This disclosure contemplates that the machine learning model can be another supervised learning model, semi-supervised learning model, or unsupervised learning model. Machine learning models are known in the art and are therefore not described in further detail herein.

A convolutional neural network (CNN) is a type of deep neural network that has been applied, for example, to image analysis applications. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks.

In some implementations, the model takes an input image of size 224×224-pixel (˜112×112 micron, 20× magnification). Although training with high-resolution images adds computational burden, it was essential to train the model with high-resolution images to capture all possible features that help classify the cells into different grades.

In some embodiments, the architecture of the GLASS-AI network is configured to classify each pixel in the input image into one of six target classes: Normal alveolar, Normal airway, Grade 1 LUAD, Grade 2 LUAD, Grade 3 LUAD, and Grade 4 LUAD. In general, the GLASS-AI network architecture consists of encoder and decoder architectures.

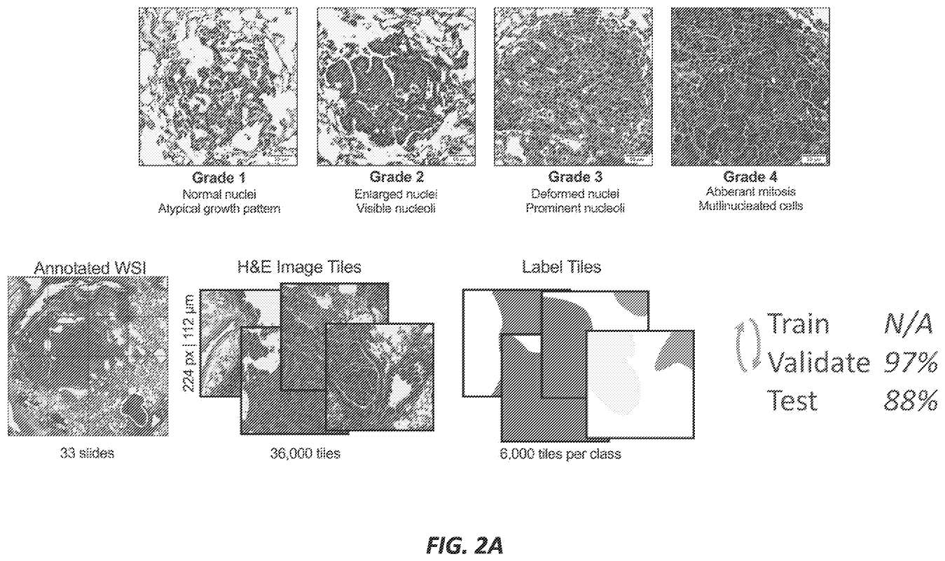

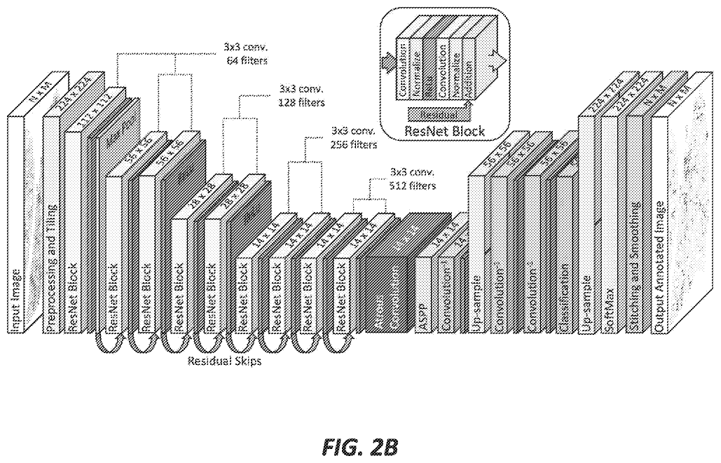

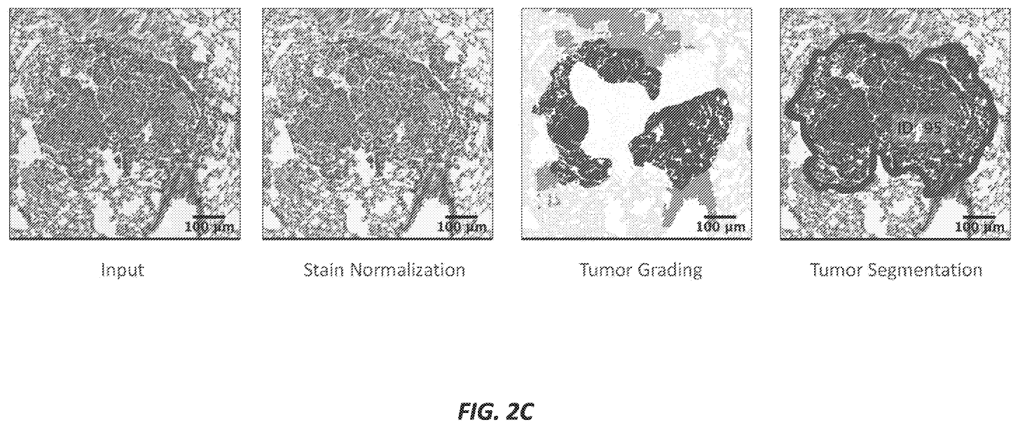

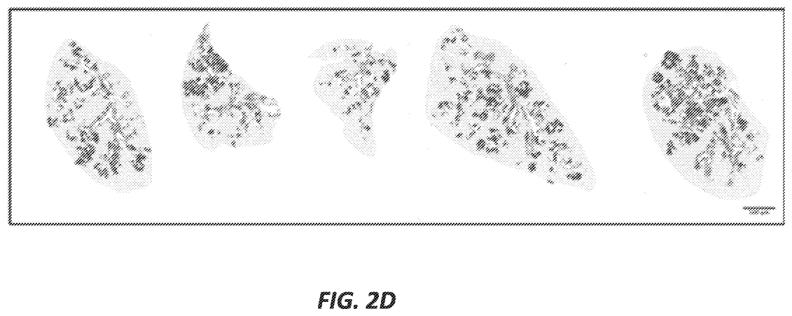

In some implementations, the supervised machine learning model is a convolutional neural network (CNN). For example, the supervised machine learning model can include one or more Residual Neural Network (ResNet) layers or components. Optionally, in examples described herein, the supervised machine learning model is ResNet-18, which is an 18-layer residual neural network that incorporates inputs from earlier layers to improve performance and is pretrained on a known dataset (i.e., the ImageNet dataset). It should be understood that ResNet-18 is provided only as an example and that other ResNet architectures may be used. There are multiple variations of ResNet architectures, such as ResNet16, ResNet18, ResNet50, and ResNet101, which may be used in different implementations. The reason for choosing 18-layer architecture as an encoder is to avoid the vanishing gradient problem that may occur when a network has deeper layers. Additionally, the residual neural network architecture can be modified to include one or more atrous convolutional layers. An atrous convolutional layer (sometimes referred to as a dilated convolutional layer) introduces a dilation rate parameter, which defines spacing between values in the kernel, to the convolution. Atrous convolutional layers are known in the art and therefore not described in further detail herein. Alternatively or additionally, the residual neural network architecture is optionally modified to include one or more transposed convolutional layers. A transposed convolutional layer (sometimes referred to as a fractionally strided convolutional layer) performs a convolution operation but reverts its spatial resolution. Transposed convolutional layers are known in the art and therefore not described in further detail herein. An example supervised learning model architecture is shown in FIG. 2B. The ResNet architecture in FIG. 2B is modified to include, among other layers, one atrous convolutional layer and a plurality (e.g., 2) of transposed convolutional layers. The atrous convolutional layer is configured to expand the field of view of the final convolutional layer in the ResNet architecture and the transposed convolutional layers are configured to further expand the output from the atrous convolutional layer such that the supervised learning model is configured to assign classifications on a pixel-by-pixel basis or cell-by-cell basis. This is as opposed to assigning classification to the digital pathology image as a whole, which would be the case solely employing a ResNet architecture. It should be understood that the supervised learning architecture shown in FIG. 2B is provided only as an example.

In some embodiments, decoder layers may consist of one or more components, including, but not limited to, parallel atrous spatial pyramid pooling layer(s), up-sampling layer(s), SoftMax layer(s), classification layer(s), and/or smoothing layer(s).

An example Parallel Atrous Spatial Pyramid Pooling (ASPP) may be configured to capture distinctive features, such as cell and nucleus size and shape, which helps differentiate between tumor grades that look very similar (e.g., differentiate between grade 3 and grade 4 cells). Therefore, the output of the ResNet18 is convolved with multiple parallel atrous convolutions containing different dilation rates. This ensures better capture of the image's multiscale contextual and semantic information.

An example up-sampling layer may be configured to classify each pixel in the input images, transposed convolution layer is used to up-scale the features maps to generate an output feature map with a spatial dimension equal to the input image.

An example SoftMax layer may be configured to utilize a SoftMax function that takes the up-sampled feature maps from the previous layer and assigns probabilities to each class.

An example classification layer may be configured to compute the cross-entropy loss for classification and weighted classification tasks with mutually exclusive classes. The layer infers the number of classes from the output size of the previous SoftMax layer.

An example smoothing layer may comprise a final layer added at the end of a plurality of layers to smooth predictions and minimize artifacts from image patch edges and produce smooth output labels.

In various examples, the GLASS-AI model classifies each pixel in the input image and produces an image labeled with the predicted classes. The final labeled image is smoothed by the last layer to remove artifacts and pixelation.

Additionally, the example method includes grading, using the artificial intelligence model, the one or more tumors within the LUAD tissue sample. The step of grading optionally includes assigning each of the one or more tumors to one of a plurality of classes. For example, the classes can include one or more of normal alveolar, normal bronchiolar, Grade 1 LUAD, Grade 2 LUAD, Grade 3 LUAD, Grade 4 LUAD, and Grade 5 LUAD. Alternatively or additionally, the step of grading includes generating graphical display data for a pseudo color map of the one or more tumors.

In some implementations, the method further includes segmenting, using the artificial intelligence model, the one or more tumors in the digital pathology image. Alternatively or additionally, the step of segmenting includes generating graphical display data for a segmentation map of the one or more tumors.

In some implementations, the method further includes analyzing the one or more tumors. The step of analyzing optionally includes counting the one or more tumors. Alternatively or additionally, the step of analyzing optionally includes characterizing an intratumor heterogeneity of the one or more tumors.

In some implementations, the method further includes performing an immuno-histochemistry (IHC) analysis of the one or more tumors. In other words, the artificial intelligence-based methods for studying LUAD tissues samples can be integrated with an immuno-histochemistry (IHC) analysis.

An example method for integrating with an artificial intelligence-based LUAD tissue sample analysis is described herein. The method includes receiving a first digital pathology image of a first LUAD tissue sample, the first digital pathology image being a hematoxylin & eosin (H&E) stained slide image; inputting the first digital pathology image into an artificial intelligence model; grading, using the artificial intelligence model, the one or more tumors within the first LUAD tissue sample; and segmenting, using the artificial intelligence model, the one or more tumors in the digital pathology image. Additionally, the method includes receiving a second digital pathology image comprising a second LUAD tissue sample, the second digital pathology image being an immuno-stained slide image; and identifying and classifying a plurality of positively and negatively stained cells within the second LUAD tissue sample. The method further includes co-registering the first and second digital pathology images; and projecting a plurality of respective coordinates of the positively and negatively stained cells within the second LUAD tissue sample onto the one or more tumors within the first LUAD tissue sample.

An example transfer learning method is also described herein. The method includes training a machine learning model with a dataset, where the dataset includes a plurality of mouse model digital pathology images. Each of the mouse model digital pathology images is of a respective lung LUAD tissue sample from a mouse. The method further includes receiving a digital pathology image of a LUAD tissue sample from a human; inputting the digital pathology image into the trained machine learning model; and grading, using the trained machine learning model, one or more tumors within the LUAD tissue sample from the human. Thus, as described herein, the machine learning model trained on mouse models is transferred to perform with acceptable accuracy in inference mode on digital pathology images of human tissue samples.

### Example Computing Device

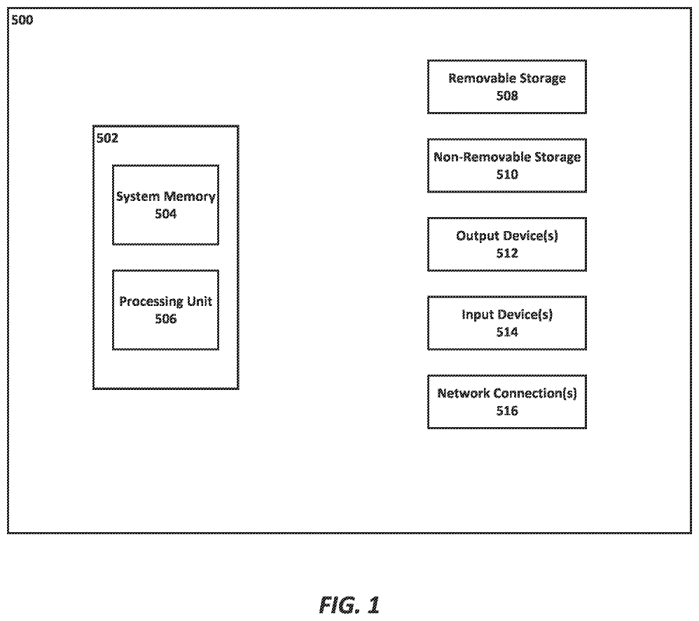

It should be appreciated that the logical operations described herein with respect to the various figures may be implemented (1) as a sequence of computer implemented acts or program modules (i.e., software) running on a computing device (e.g., the computing device described in FIG. 1), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 1, an example computing device 500 upon which the methods described herein may be implemented is illustrated. It should be understood that the example computing device 500 is only one example of a suitable computing environment upon which the methods described herein may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random-access memory (RAM)), non-volatile (such as read-only memory (ROM), flash memory, etc.), or some combination of the two. This most basic configuration is illustrated in FIG. 1 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Embodiments of the present disclosure provide a novel, open-source tool for the research community using a machine learning-based pipeline for grading histological cross-sections of lung adenocarcinoma in mouse models. In addition, the machine learning model uncovers a significant degree of intratumor heterogeneity that is not reported by human raters.

Preclinical mouse models of lung adenocarcinoma are invaluable for investigating molecular drivers of tumor formation, progression, and therapeutic resistance. However, histological analysis of these preclinical models requires significant time and training to ensure accuracy and consistency. To achieve a more objective and standardized analysis, deep learning was used to create GLASS-AI (Grading of Lung Adenocarcinoma with Simultaneous Segmentation by Artificial Intelligence), a histological image analysis tool that can be widely utilized by the research community to grade, segment, and analyze tumors in mouse models of lung adenocarcinoma. GLASS-AI demonstrates strong agreement with expert human raters while uncovering a significant degree of unreported intratumor heterogeneity. Integrating immunohistochemical staining with high-resolution grade analysis by GLASS-AI identified dysregulation of Mapk/Erk signaling in high-grade lung adenocarcinomas and locally advanced tumor regions. The present disclosure demonstrates the benefit of employing GLASS-AI in preclinical lung adenocarcinoma models and the power of integrating machine learning and molecular biology techniques for studying cancer progression. GLASS-AI is available from https://github.com/jlockhar/GLASS-AI.

The approval of whole slide scanners for use in clinical pathology by the U.S. Food and Drug Administration (FDA) in 2017 led to the rapid proliferation of digital pathology images in both healthcare and pre-clinical settings. Not only have whole slide images (WSIs) increased the efficiency of pathologists' workflow, but their digitization also enables collaboration among geographically distant groups. Furthermore, advances in computer vision and image processing have given rise to several applications that can assist in the histopathological analysis of WSIs, particularly in the field of oncology. These applications often utilize pre-trained convolutional neural networks (CNNs) to perform or assist with time-consuming tasks, such as nuclei segmentation^{1,2} histological staining analysis³, and tumor segmentation⁴⁻⁶. Similar machine learning approaches have been developed for more nuanced analyses, including quantifying tumor-associated or tumor-infiltrating immune cells⁷⁻⁹, microsatellite instability¹⁰, and prediction of patient mutational status from WSIs^{11,12}. Machine learning models trained to classify tumors into diagnostically distinct grades using existing systems, such as Gleason score for prostate cancer¹³⁻¹⁵, have also been reported. In many of these studies, the accuracy of the machine learning model has been measured in terms of agreement with expert human raters on a sample-by-sample basis. While a suitable measure of performance, this comparison level fails to capture much of the information uncovered by the high-resolution analysis these algorithms perform.

In addition, the development of these machine learning models has been focused almost exclusively on analyzing human samples. For clinical applications, building human-focused models from observational data from human patients, like that stored in The Cancer Genome Atlas (TCGA)'s collection of WSIs and the associated molecular data¹⁶, is ideal. However, the intense focus on clinical applications has provided very few machine learning models useful for translational and basic research. Machine learning applications in pre-clinical research present an excellent opportunity to enhance and accelerate analyses of the experimental data produced from these sources.

Several mouse models of lung adenocarcinoma (LUAD) have been reported, of which the Kras^{LSL-G12D/+} model is the most widely used¹⁷. This well-studied model serves as a valuable baseline for studying other mutations commonly found in LUAD, such as Trp53^{R172H}, separately or in conjunction with the activating Kras^{G12D} mutation. These models often develop over 100 primary tumors, making a thorough analysis of these valuable specimens extremely time-consuming, even for experienced researchers.

Embodiments of the present disclosure provide GLASS-AI (Grading of Lung Adenocarcinoma with Simultaneous Segmentation by Artificial Intelligence), a machine learning pipeline for the analysis of mouse models of lung adenocarcinoma that provides a rapid means of analyzing tumor grade from WSIs. The GLASS-AI pipeline was trained on multiple genetically engineered mouse models to ensure that it generalized well. Analysis of several mouse models of LUAD revealed a high degree of accuracy, comparable to expert human raters. Furthermore, the high-resolution analysis performed by GLASS-AI revealed extensive intratumor heterogeneity that was not reported by the human raters. Alignment of these heterogeneous tumor regions with adjacent immunostained sections showed a strong correlation between tumor grade and aberrant Mapk/Erk signaling that differed between Kras^{G12D/+} (K), TAp73^{Δ/Δ}; Kras^{G12D/+} (TK), and Kras^{G12D/+}. Trp53^{R172H/+} (KP) mouse models. The GLASS-AI pipeline empowers pre-clinical research by rapid analysis of LUAD without the need for extensive training of human raters.

### Training of Machine Learning Model

Developing an accurate machine learning model requires a large amount of high-quality training data. To construct a training dataset, WSIs were collected from Kras^{G12D/+}. Rosa^{mG/mG} (K) (n=4), TAp73^{Δ/Δ}; Kras^{G12D/+} (TK) (n=15), and Kras^{G12D/+}; Trp53Δ/Δ (n=14) mice 30 weeks after LUAD initiation. Slides were divided among three expert human raters who segmented and graded tumors using the Grade 1-Grade 5 scale reported by Tyler Jacks' laboratory^{18,19}, although Grade 5 areas were not observed within the animals used for generating the training library. The WSIs were then divided into 224×224-pixel (approximately 112×112 micron) image patches and corresponding annotation patches (FIG. 2A). The final training library comprised approximately 6,000 patches for each of the six target classes (Normal alveolar, Normal airway, Grade 1 LUAD, Grade 2 LUAD, Grade 3 LUAD, and Grade 4 LUAD) and was split 60/20/20 for model training, validation, and testing (FIG. 2A). Data augmentation was used to ensure that each of the target classes was equally represented within the training, validation, and testing datasets.

In the example depicted in FIG. 2B, the machine learning model was based on ResNet18²⁰ with a rectified linear unit (ReLU)-only pre-activation. The outputs of GLASS-AI were designed to produce graphical maps of tumor grading calls (FIG. 2C, middle) and segmented tumors (FIG. 2C, right) in addition to tabulation of areas of each grade within each segmented tumor and the whole image. WSIs can be input directly into GLASS-AI for analysis. (FIG. 2D). Several optional settings are included in the standalone application developed to run GLASS-AI on Windows or Macintosh computers, including digital stain normalization, a low memory mode to facilitate analysis of large files like WSIs, and customization of various parameters used for tumor segmentation and output generation.

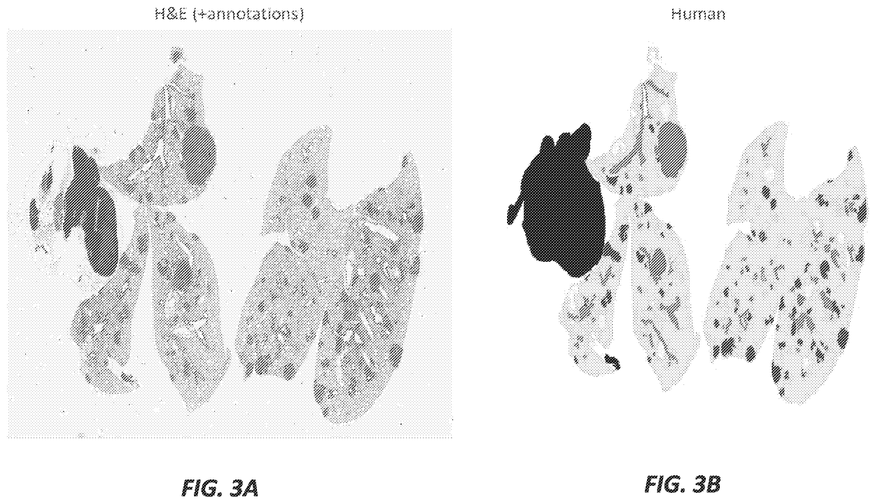

Referring now to FIGS. 3A-B, schematic diagrams in accordance with certain embodiments of the present disclosure are provided. In particular, FIG. 3A depicts an example of human annotations on a H&E image while FIG. 3B depicts the class masks generated from the annotations. In FIGS. 3A-B, Cyan=Normal alveoli, Magenta=normal airway, Green=Grade 1 LUAD, Blue=Grade 2 LUAD, Yellow=Grade 3 LUAD, Red=Grade 4 LUAD, Black=Ignore. The lymphoid structure on the left of the slide was masked out by the human rater since GLASS-AI has no output class that would be correct for those structures.

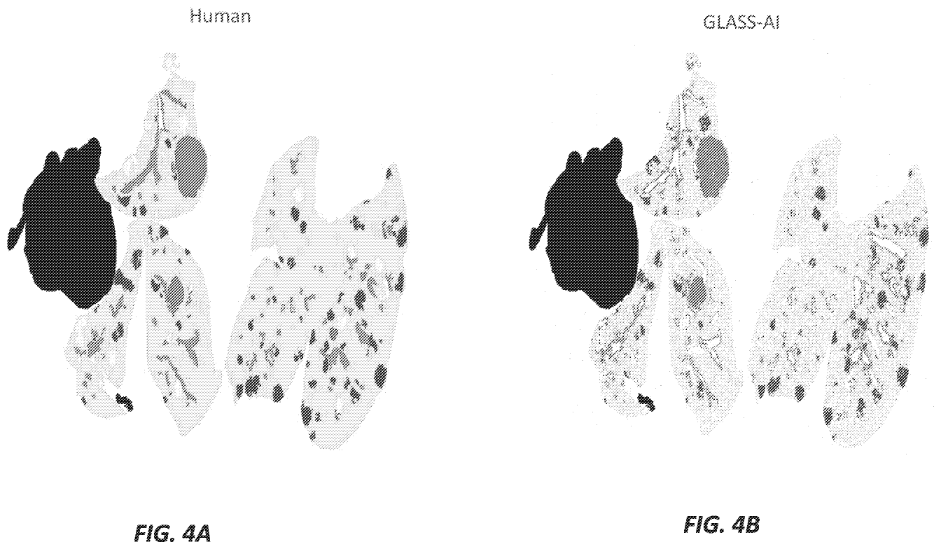

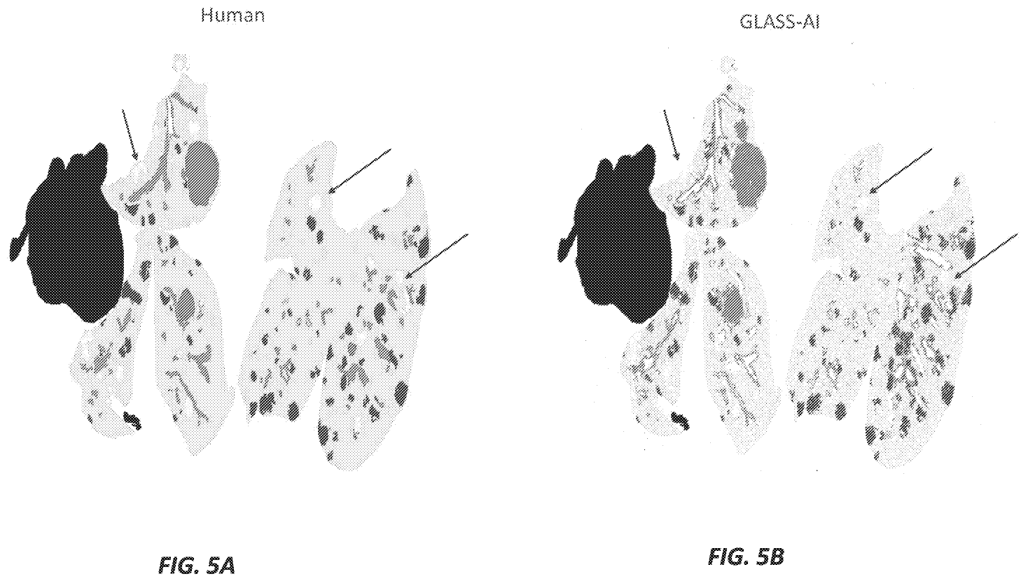

Referring now to FIGS. 4A-B, schematic diagrams in accordance with certain embodiments of the present disclosure are provided. In particular, FIG. 4A depicts human annotations and FIG. 4B depicts GLASS-AI's corresponding output. GLASS-AI excludes empty space/voids, so the empty area of the alveoli and airways is visible. The human annotations fill in these empty spaces as manually segmenting them is not feasible. Arrows shown in FIGS. 4A-B indicate tumors that demonstrate small regions of higher grade that will result in the tumor being called as a higher grade when an overall tumor grade is assigned using the output from GLASS-AI, as further depicted in more detail in FIGS. 5A-B.

Referring now to FIGS. 5A-B, schematic diagrams in accordance with certain embodiments of the present disclosure are provided. In particular, FIGS. 5A-B depict a comparison of the human annotations (FIG. 5A) and GLASS-AI's output (FIG. 5B). GLASS-AI excludes empty space/voids, so the empty area of the alveoli and airways is visible. The human annotations fill in these empty spaces as manually segmenting them is not feasible. Arrows indicate tumors that demonstrate small regions of higher grade that will result in the tumor being called as a higher grade when an overall tumor grade is assigned using the output from GLASS-AI. The lymphoid structure on the left of the slide was masked out by the human rater since GLASS-AI has no output class that would be correct for those structures.

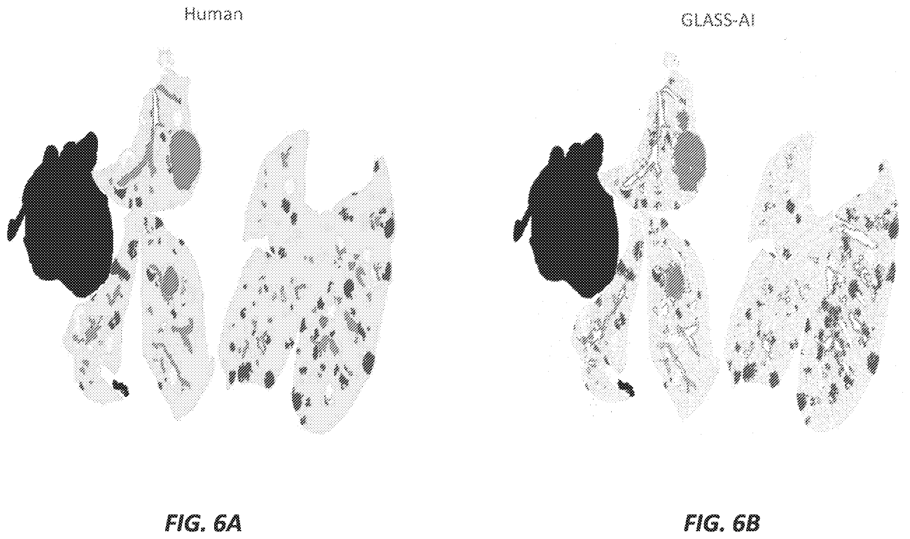

Referring now to FIGS. 6A-B, schematic diagrams in accordance with certain embodiments of the present disclosure are provided. In particular, FIGS. 6A-B depict another comparison of human annotations (FIG. 6A) and GLASS-AI's output (FIG. 6B). Arrows indicate tumors that demonstrate small regions of higher grade that will result in the tumor being called as a higher grade when an overall tumor grade is assigned using the output from GLASS-AI.

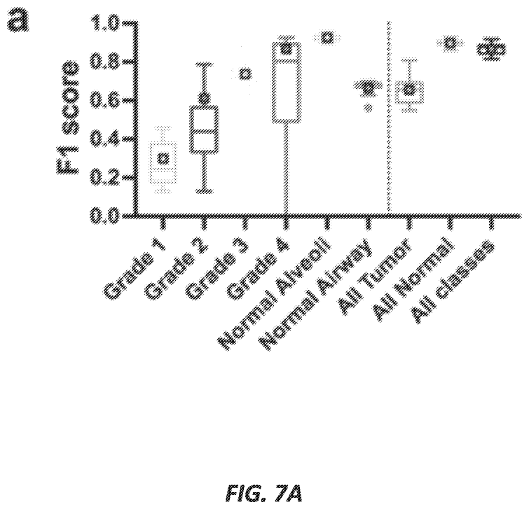

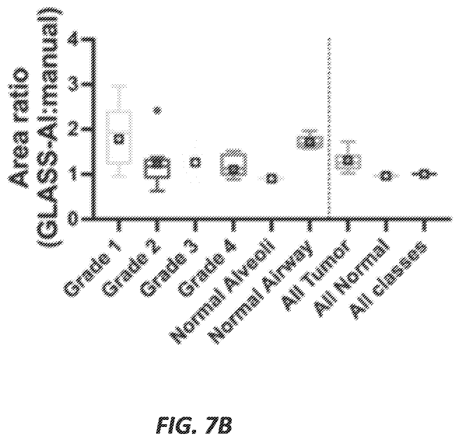

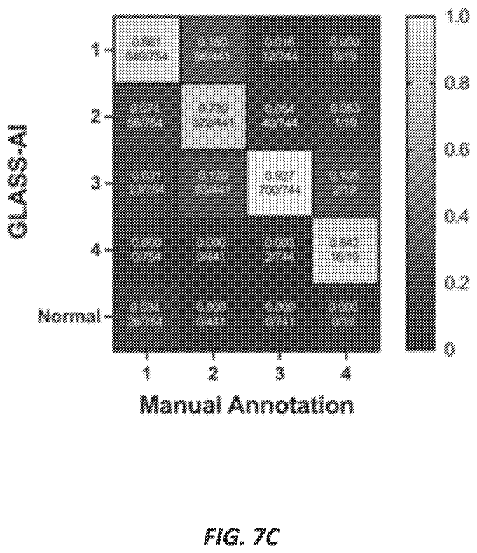

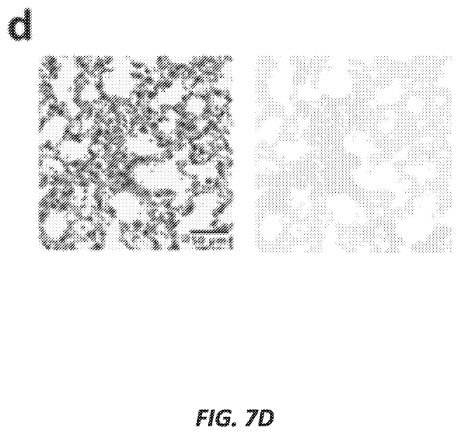

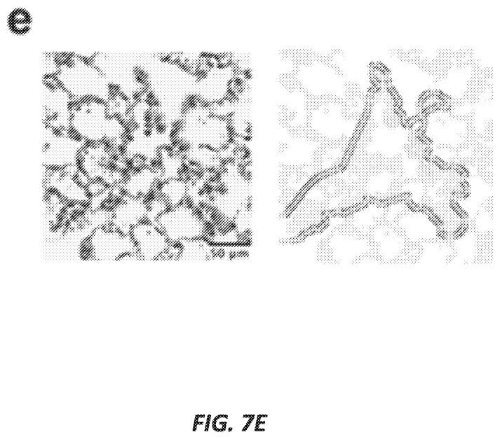

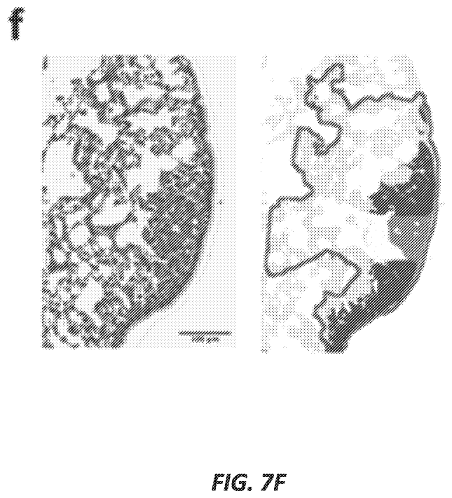

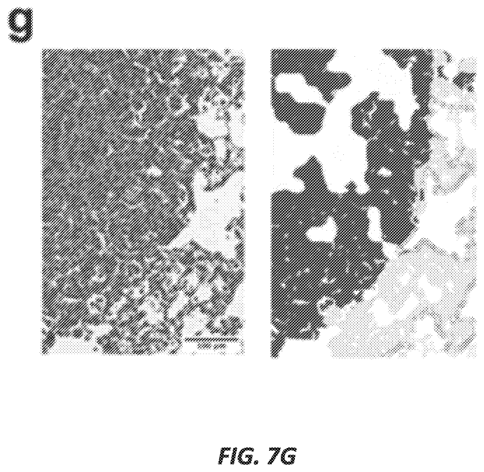

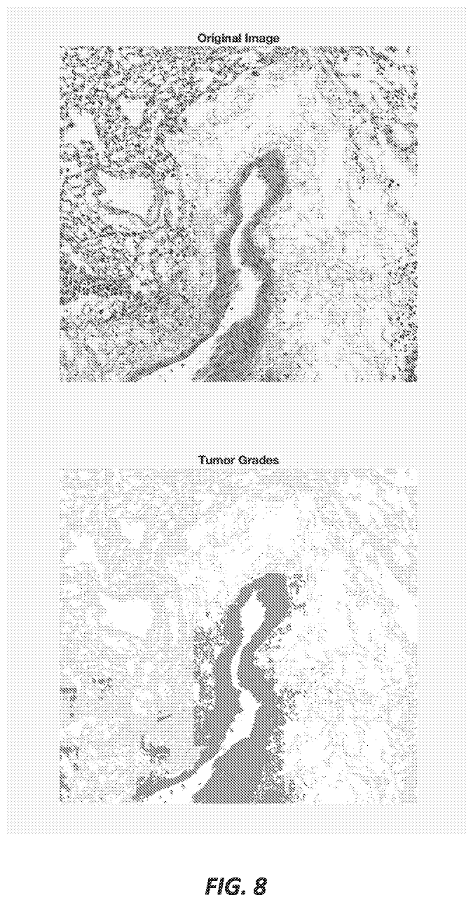

After training, GLASS-AI achieved an accuracy of 88% on the patches in the final testing data set. However, the image patches used in this assessment do not entirely capture segmentation and classification accuracy due to their small size and disconnected nature. Therefore, the performance of GLASS-AI is compared against another human rater on a group of 10 complete WSIs within which a total of 1958 tumors were manually segmented and graded. After assigning a single grade to each tumor segmented based on the highest tumor grade that comprised at least 10% of the tumor's area, GLASS-AI achieved a Micro F1-score of 0.867. Examining the F1-score for each class showed a trend toward higher a higher score with increasing tumor grade (FIG. 7A). By comparing the ratio of the tumor areas annotated by GLASS-AI and the human rater, it was discovered that GLASS-AI annotated an average of 31% more tumor area. This increase was most pronounced in the Grade 1 tumors (FIG. 7B), which are usually smaller and more difficult to notice compared to tumors of higher grades. Reviewing the Grade 1 areas identified by GLASS-AI and not the human rater showed that a number of these regions were likely Grade 1 LUAD or atypical adenomatous hyperplasia that were missed by the human rater (FIG. 7D). A large increase in the amount of normal airway area found by GLASS-AI was also observed. Upon inspection, it was found that this was due to misclassification of the smooth muscle cells of the pulmonary arteries, a cell type also present surrounding the airways of the lung as depicted in FIG. 8.

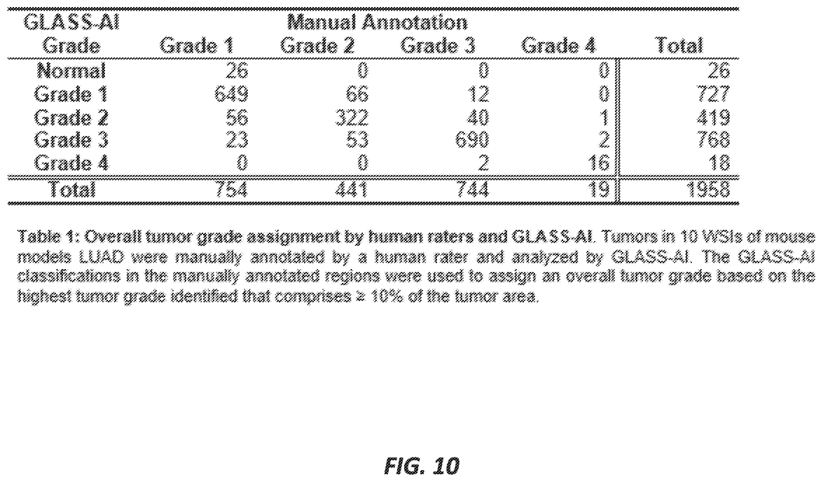

Overall, GLASS-AI successfully recognized tumors within 1932 of the 1958 manually segmented regions as depicted in FIG. 10 (Table 1). All 26 of the tumors missed by GLASS-AI were manually annotated as Grade 1 and were classified as “normal alveoli” (FIG. 7E). In addition to identifying 98.8% of manually annotated tumors, GLASS-AI's grading also covered 90% of the manually annotated tumor area.

To better compare tumor grading between GLASS-AI and the human rater, the manually annotated regions were used in combination with GLASS-AI's grading to assign the tumor grade. GLASS-AI and the human rater assigned the same grade to 1380 (85.7%) of the annotated tumors resulting in a Cohen's kappa of 0.760. It was observed that the grading agreement was high across all 4 tumor grades (FIG. 7C). As shown in FIG. 3C, the GLASS-AI output covered 98.8% of manually annotated tumors (1932/1958), 90% of manually annotated tumor area, and assigned the same overall grade in relation to 86% of tumors. It was observed that some tumors that received discordant grades contained regions of higher grade did not reach the threshold of 10% of the tumor area to define the tumors grade (FIG. 7F). However, it was clear that the grade assignment system was capable of handling heterogenous tumors (FIG. 7G).

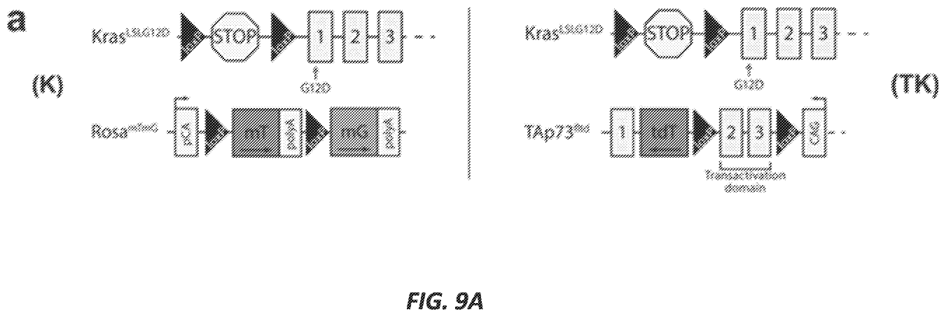

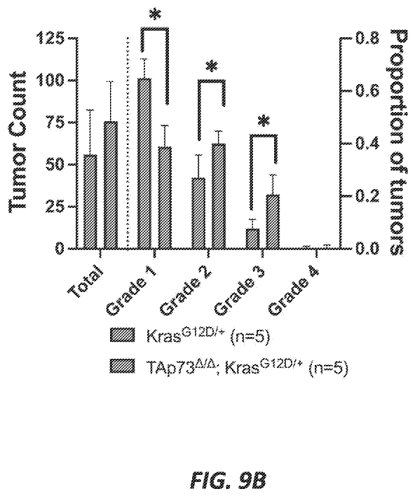

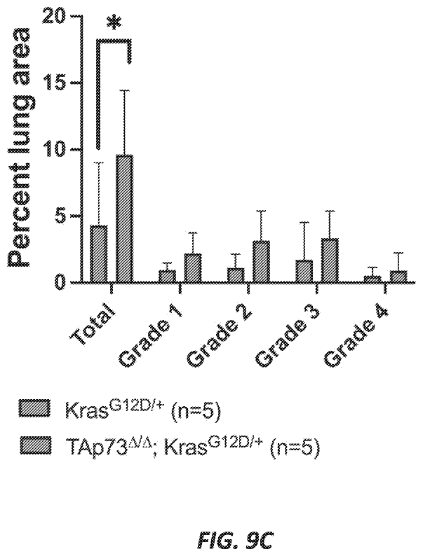

Referring now to FIGS. 9A-D, schematic diagrams depicting manual histological characterization of lung adenocarcinoma mouse models is provided. FIG. 9A provides a schematic representation of mutant alleles in K and TK mouse models. FIG. 9B and FIG. 9C provide graphs depicting Five K and TK mice (n=5) analyzed by an expert human rater, segmenting and grading each tumor in the lung section. The number of total tumors and tumors of each grade (FIG. 9B) along with tumor burden (FIG. 9C) was compared between the K and TK mice and analyzed by Student's t-test with Holm-Sǐdák correction. Data represents mean±SD. *p<0.05 for the indicated comparison between K and TK mice.

### GLASS-AI Analysis of Mouse Models of Lung Adenocarcinoma.

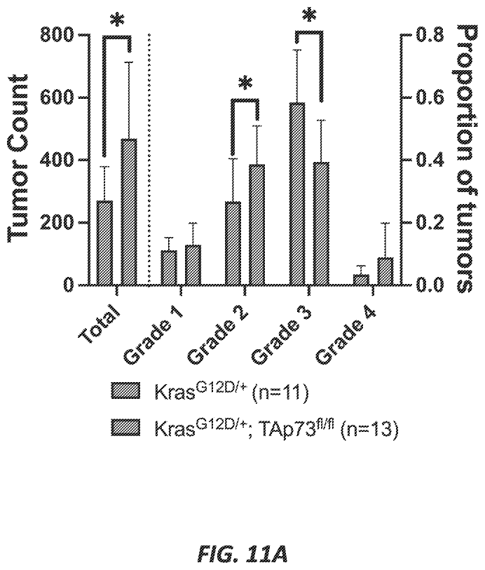

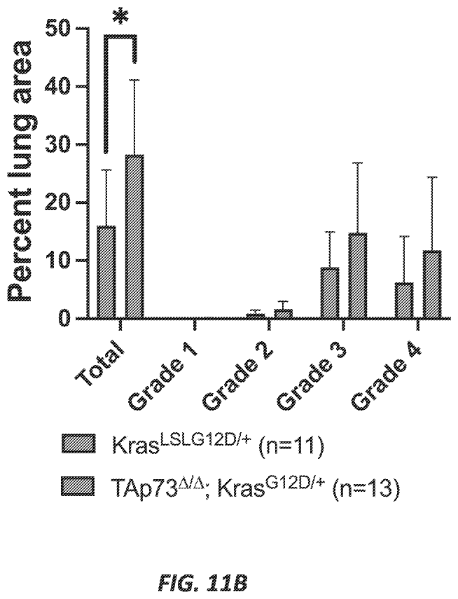

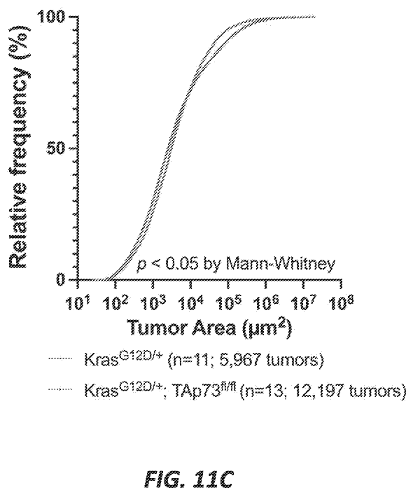

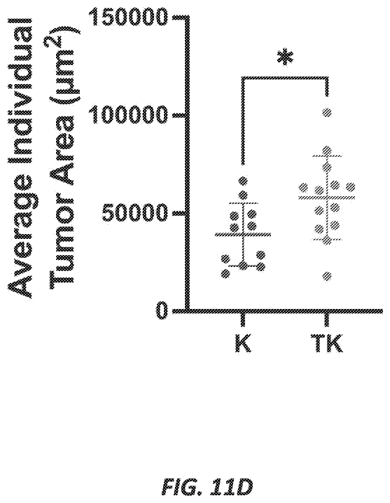

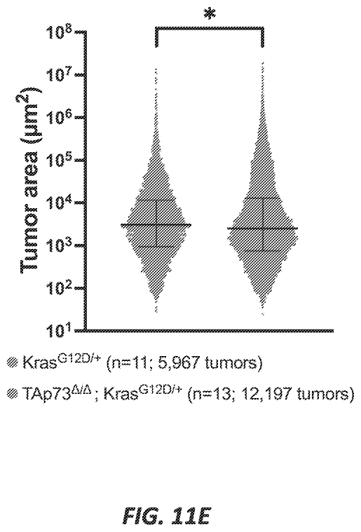

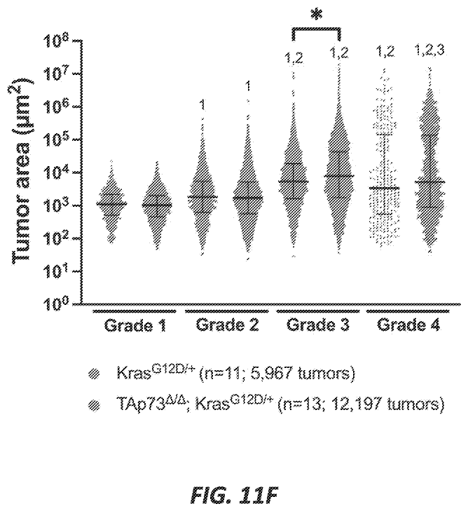

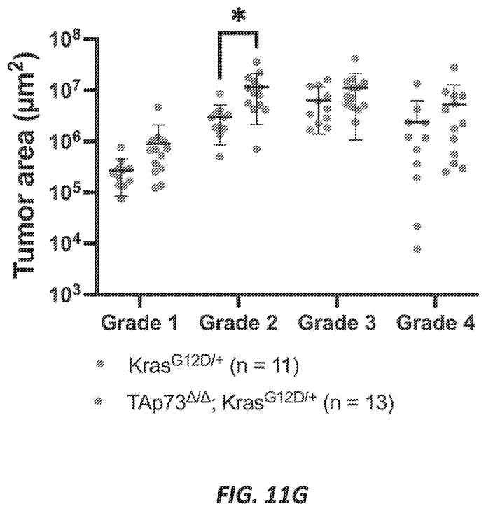

The initial test of the GLASS-AI pipeline was carried out on Kras^{G12D/+}; Rosa^{mG/mG} (K) and TAp73^{Δ/Δ}; Kras^{G12D/+} (TK) mouse models (FIG. 11A). The tumor phenotypes of the K mouse model have been characterized by previous studies^{17,19,21} while the tumor phenotypes in the TK model are currently under investigation. The mice used for these studies were collected 30 weeks after initiation of LUAD by intratracheal instillation with adenovirus expressing Cre recombinase under the control of a CMV promoter.

After analyzing a cohort of 11 K and 13 TK mice with GLASS-AI, it was found that the number of tumors in TK mice was significantly higher than in K mice (FIG. 11B). When the grading algorithm used by human raters (i.e., highest tumor grade with ≥10% tumor area) was applied to the tumors identified by GLASS-AI, it was observed that the distribution of tumors by grade in K mice differed from the manual analysis; the majority of tumors in the K mice were Grade 3 with a few Grade 1 or Grade 4 tumors. However, the TK mice exhibited a significant decrease in the proportion of tumors rated as Grade 3 compared to K mice. This shift away from Grade 3 was accompanied by a significant increase in the proportion of Grade 2 and Grade 4 tumors (FIG. 11B). In addition, the proportion of lung area occupied by tumors annotated by GLASS-AI in TK mice was significantly higher than in K mice. TK mice exhibited an increase in the percentage of lung area filled by each grade, particularly Grade 3 and Grade 4 LUAD (FIG. 11C). It was found that the tumors of TK mice were approximately 1.5-fold larger (p<0.05) than the tumors of K mice as depicted in FIG. 11D. From these data, it can be concluded that the loss of TAp73 drives tumor formation, increases tumor burden, and alters tumor progression.

The distribution of individual tumor sizes was examined to determine if the increased tumor burden observed in TK mice compared to K mice was due solely to an increased tumor number. Interestingly, while the median tumor size of TK mice was found to be significantly smaller than K mice as depicted in FIG. 11E, a closer examination of the cumulative distribution of tumors revealed that TK mice had a broader distribution of tumor sizes with a higher proportion of smaller and larger tumors than K mice (FIG. 11F). However, the 6-log range of tumor sizes observed meant that the more numerous small tumors of the TK mice contributed relatively little to the overall tumor burden. Indeed, 50% of the total tumor area was contained in only 30 (0.5%) and 125 (1.0%) tumors from K and TK mice, respectively. Looking at the distribution of tumors of each grade in the K and TK mice, it was observed that tumors of higher grades were significantly larger than lower grade tumors. Furthermore, it was noted that Grade 3 tumors from TK mice were also significantly larger than those of K mice (FIG. 11E). Interestingly, when the area of each grade was examined on a pixel-by-pixel rather than tumor-by-tumor basis, it was found that TK mice had a significant expansion of Grade 2 area compared to K mice. In some embodiments, the area of each grade may be examined directly on a pixel-by-pixel basis using the grading performed by GLASS-AI rather than the estimated overall tumor grade calculated from the output of GLASS-AI. This expansion was proportionally larger than the Grade 3 and Grade 4 expansions, which did not reach statistical significance as depicted in FIG. 11G. Therefore, it can be concluded that the expansion of tumors seen in TK mice is due, at least in part, to an increase in the Grade 2 tumor area.

### Uncovering Intratumor Heterogeneity.

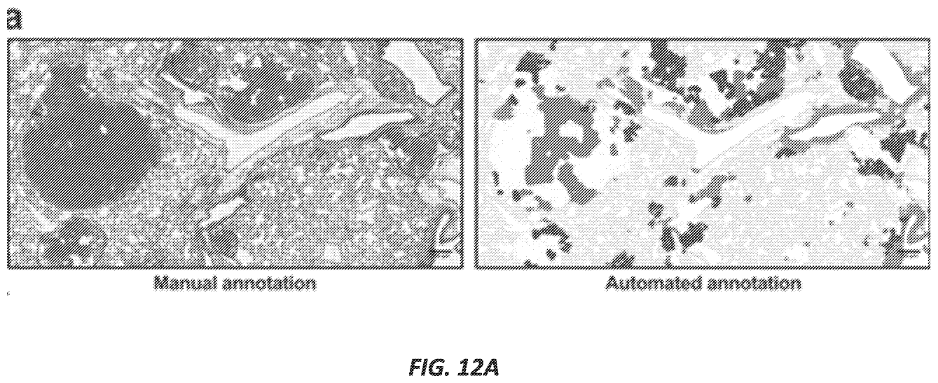

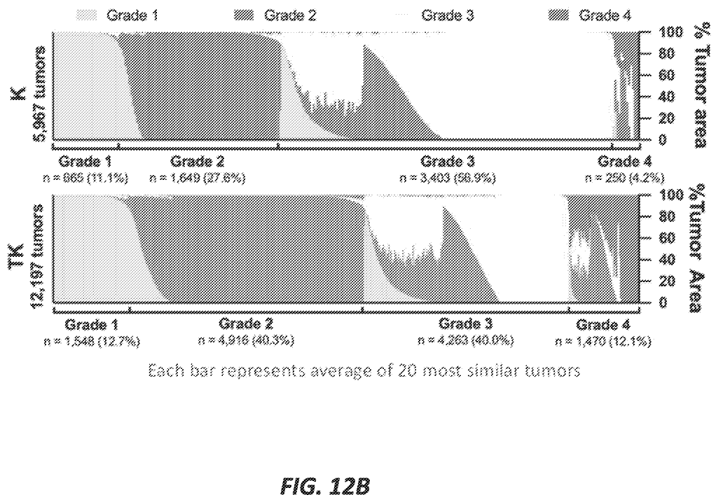

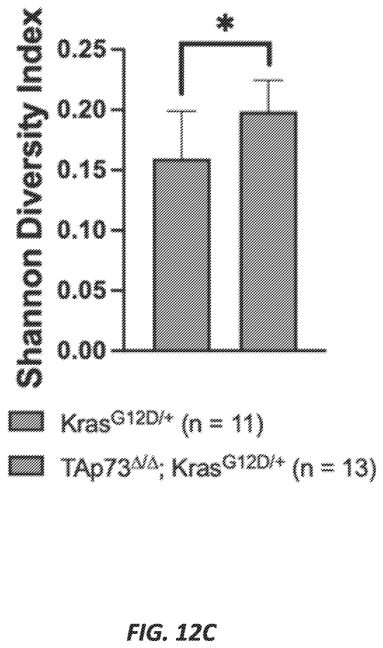

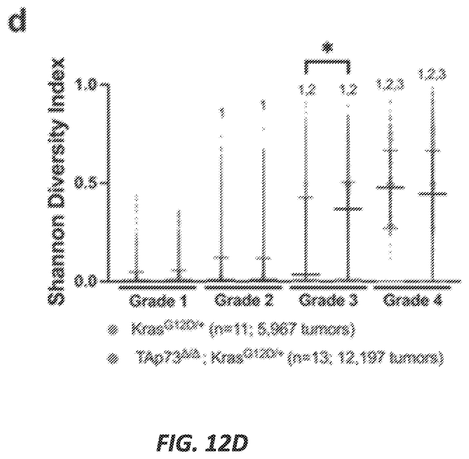

FIG. 12A and FIG. 12B provide schematic diagrams depicting an example of human annotations (FIG. 12A) compared with a GLASS-AI output (FIG. 12B) that highlights the intratumor heterogeneity that is uncovered by GLASS-AI and obfuscated by the manual annotation. In particular, FIG. 12B depicts a heatmap of tumor grade agreements between a human rater and GLASS-AI on a set of 10 whole slide images (5 Kras^{G12D/+}, 5 Kras^{G12D/+}; TAp73^{Δ/Δ}).

It is important to note that the annotations generated by the expert human raters were based on standard criteria for tumor grading, in which a tumor is assigned a single grade based on the highest grade observed that comprises at least 10-20% of the tumor area 9. However, GLASS-AI gave grades to individual pixels within the image before tumor segmentation, producing a mosaic of grades within a single tumor (FIG. 12A). This intratumor heterogeneity likely decreased the accuracy measurement of GLASS-AI during training. However, this information can be used to understand better the effects of genes of interest and drivers of tumor progression in mouse models of LUAD, including the loss of TAp73 in the LUAD mouse models.

By representing each tumor as a stacked bar divided by the proportion of the tumor area made up of each grade of LUAD, the overall distribution of intratumor heterogeneity in the LUAD mouse models can be visualized (FIG. 12B). From these graphs, patterns in tumor composition can be identified, such as the relatively small proportion of Grade 1 area found in Grade 4 tumors or the presence of Grade 2 area in tumors of a higher grade. The shift from predominantly Grade 3 tumors in K mice to other tumor grades, namely Grade 2 and Grade 4, in TK mice was also evident from these graphs (FIG. 12B).

While informative, these visual representations of tumor heterogeneity can provide only a qualitative estimation of heterogeneity in the mouse models. To overcome this shortcoming, the Shannon Diversity Index (SDI) was employed as a quantitative estimate of intratumor heterogeneity. SDI estimates the uncertainty in predicting the grade of a given square micron in a tumor given by H′=−Σᵢ₌₁⁴ pᵢ In pᵢ, where p is the proportion of the i-th grade from Grade 1 to Grade 4. After estimating the mean SDI from each tumor in a mouse, it was found that the TK mice had a higher overall SDI than K mice (FIG. 12C). The individual tumors of each grade in K and TK mice were also compared and a significant trend of increasing heterogeneity with increasing tumor grade was observed (FIG. 12D). Furthermore, the Grade 3 tumors of K mice had a significantly lower SDI than TK mice. These data indicate that the loss of TAp73 increases the intratumor heterogeneity, perhaps due to the accumulation of other mutations and defects during tumor progression.

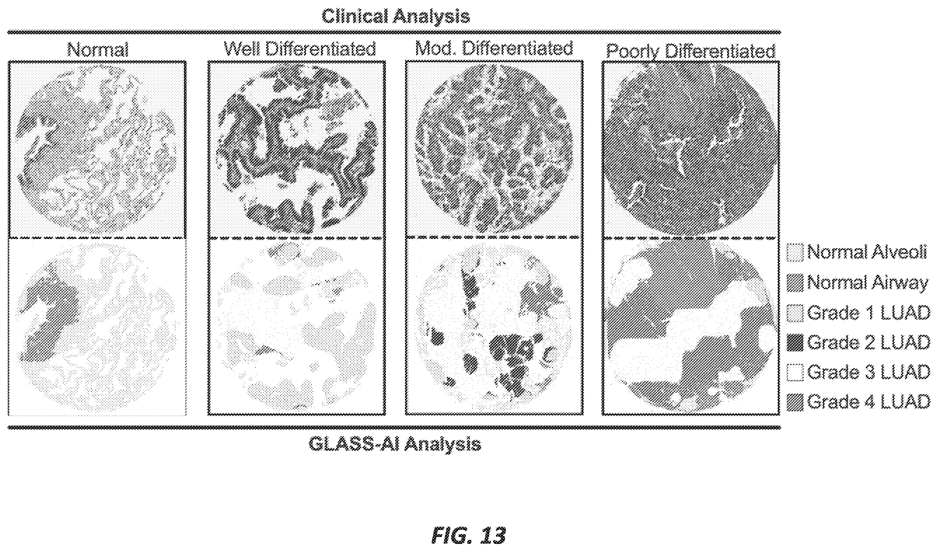

Referring now to FIG. 13, a schematic diagram depicting representative examples of cores from a human LUAD patient tissue microarray generated at Moffitt Cancer Center (“TMA 5-2”, provided by Dr. W. Douglas Cress) with the associated clinical grading (top) and GLASS-AI outputs (bottom) is provided.

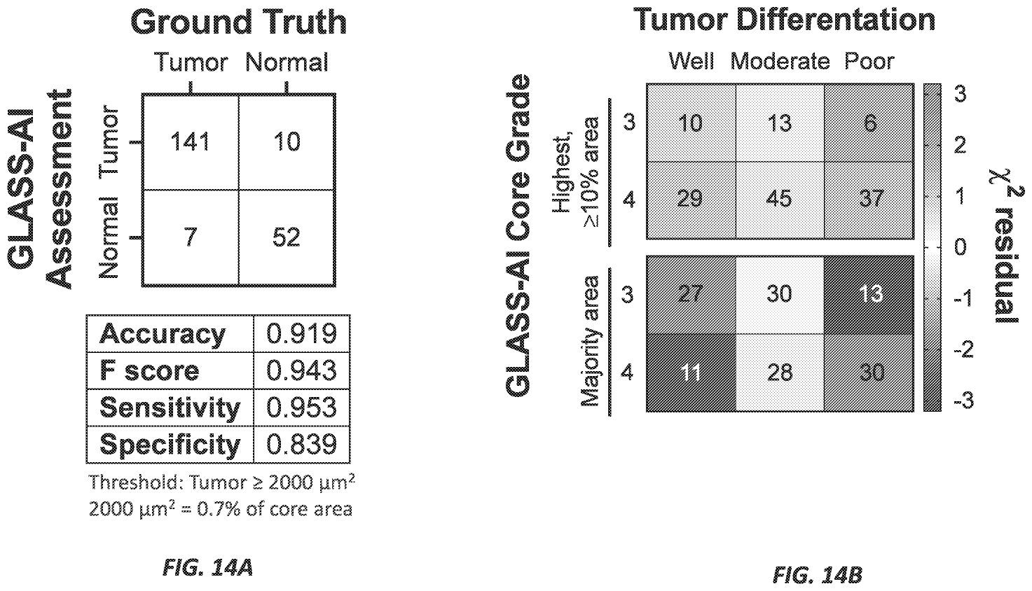

Referring now to FIGS. 14A-B, schematic diagrams depicting confusion matrices of core type calls and accuracy measures from the GLASS-AI assessment compared to clinical ground truth are provided. Tumor Microarray (TMA) core cross sections were called as “tumor” if they contained ≥2000 micron2 of tumor annotated by GLASS-AI as illustrated in FIG. 14A. As illustrated in FIG. 14B, core cross-section grades assigned by GLASS-AI using the indicated strategy compared to clinical grading performed at the time of specimen collection were compared using an omnibus Chi-squared test followed by pairwise comparisons. Labels in each square represent the number of TMA cross sections, and squares are shaded to represent Pearson residuals. When using the majority assigned as Grade 1 and was excluded from the analysis.

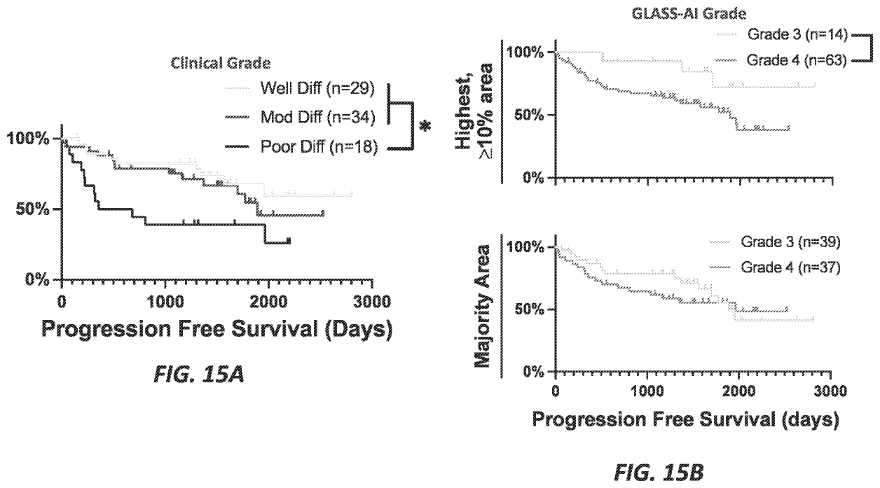

Referring now to FIGS. 15A-B, graphs depicting progression-free survival after surgical intervention are provided. The progression-free survival of patients that received only surgical intervention was analyzed based on clinical grade as depicted in FIG. 15A, or the grade assigned by GLASS-AI using the indicated strategy, as depicted in FIG. 15B. Survival curves were compared by pairwise Gehan-Breslow-Wilcoxon tests. The total number of patients in the survival curves based on GLASS-AI grading differs from the clinical grade due to the 7 false negatives predicted by the pipeline that was used. *p<0.05 for the indicated comparison.

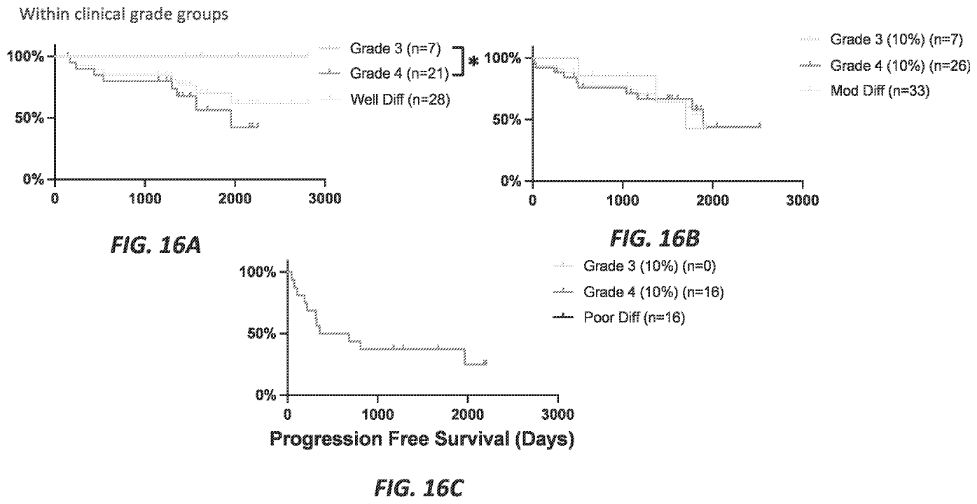

Referring now to FIGS. 16A-C, graphs depicting progression-free survival curves of patients included in the Moffitt LUAD TMA that underwent only surgical intervention, grouped by clinical grading with further stratification by GLASS-AI are provided. Notably, clinically “Well Differentiated” patients with GLASS-AI Grade 3 showed better outcomes than those with GLASS-AI Grade 4 as shown in FIG. 16A. In addition, all clinically “Poorly Differentiated” patients were assessed as Grade 4 by GLASS-AI as depicted in FIG. 16C. Overall, these results show that there may be prognostic benefits from the current clinical grading system, GLASS-AI, and a combination of the two.

### Integrating GLASS-AI and Immunohistochemistry

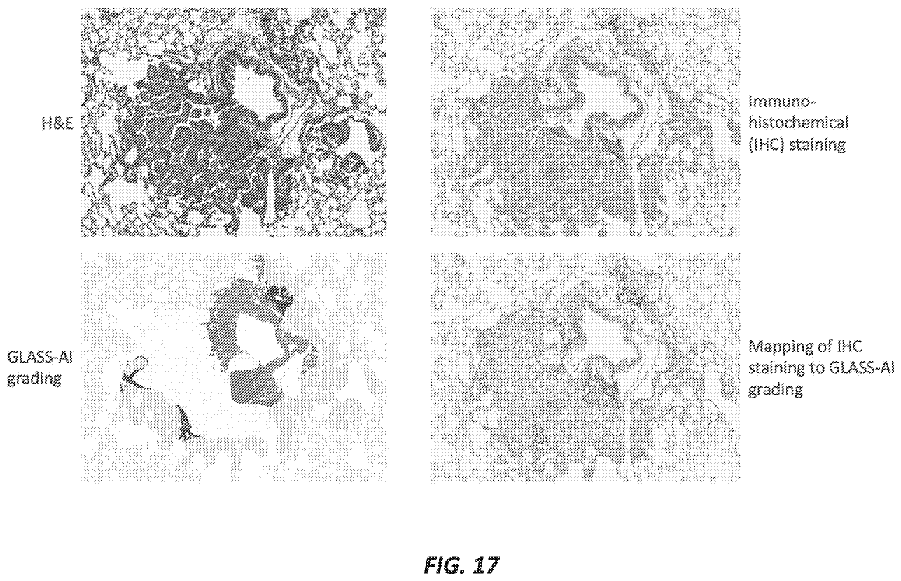

Referring now to FIG. 17, a schematic diagram depicting a demonstration of the integration of GLASS-AI with immunohistochemical analysis of adjacent slides that are registered (aligned) to the graded H&E slide is depicted.

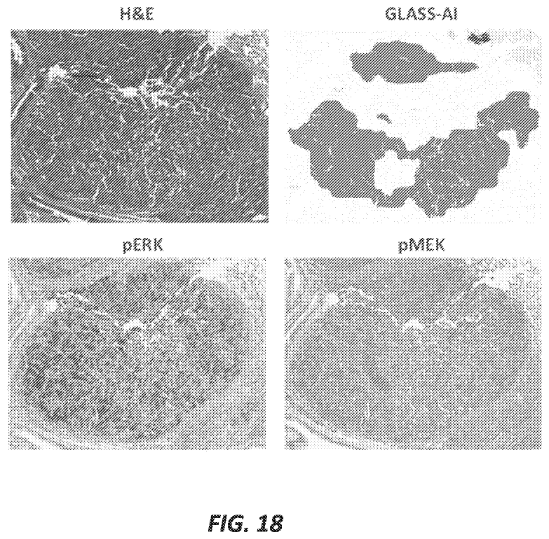

Referring now to FIG. 18, schematic diagrams in accordance with certain embodiments of the present disclosure are provided. In particular, as shown, GLASS-AI demonstrated that MEK/ERK signaling becomes increasingly dysregulated as LUAD tumors progress in multiple mouse models. Further analysis showed that the positively stained cells were enriched within high-grade regions of lower-grade tumors that were p-MEK or p-ERK positive.

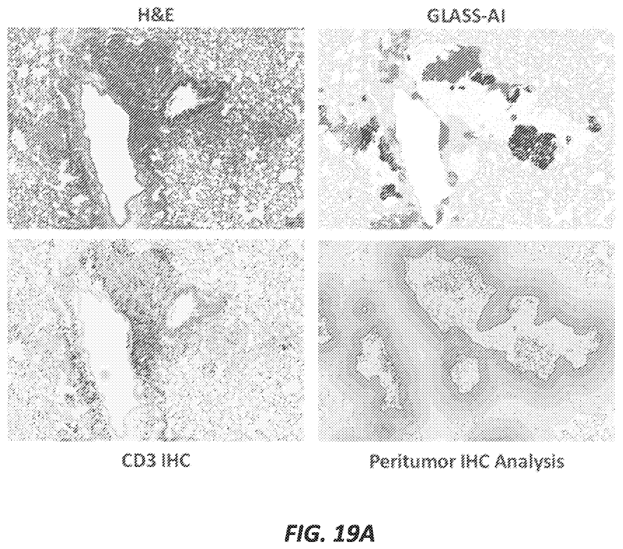

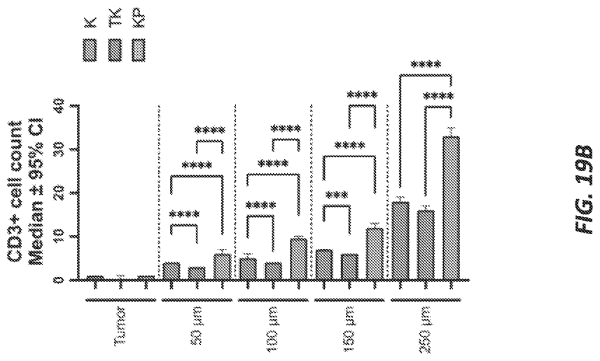

Referring now to FIGS. 19A-B, schematic diagrams in accordance with certain embodiments of the present disclosure are provided. In particular, FIGS. 19A-B illustrate that the tumor segmentations produced by GLASS-AI can be used to demarcate peri-tumor regions for IHC analysis. In the example depicted in FIG. 19A, CD3+ T-cells were stained for, which showed no significant differences within the tumors of the three mouse models tested. However, a significant difference in the recruitment of these T-cells to the peritumor regions is noted among the 3 genotypes. In particular, the decrease in CD3+ T-cells in the TK vs the K mice has been seen by the lab using other analyses, including manual IHC analysis and single cell RNAseq.

Dysfunctional Mek/Erk Signaling is Associated with Grade 4 Regions in High-Grade Tumors.

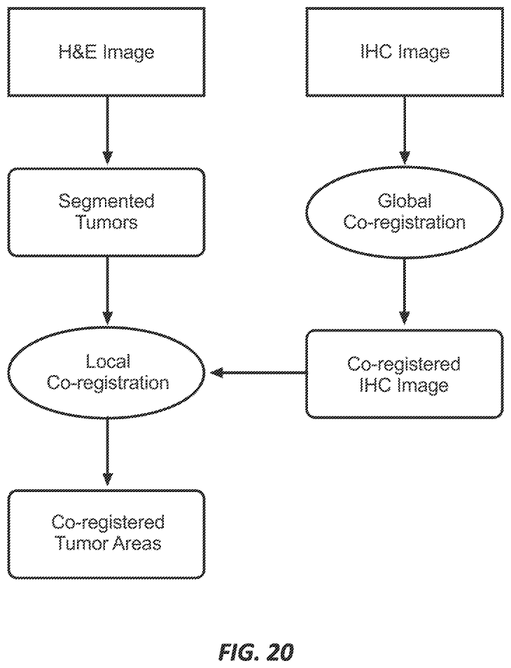

To investigate how the loss of TAp73 contributes to tumor progression and to correlate tumor grade with molecular indicators of progression, immunohistochemistry (IHC) was performed for phospho-Mek (p-Mek) and phospho-Mapk/Erk (p-Erk) on tissue sections from K and TK mouse lungs with adjacent H&E sections graded by GLASS-AI. Global and local registration on the IHC WSI were performed to ensure the highest accuracy between the H&E and IHC sections as depicted in FIG. 20. The IHC images were registered to the corresponding H&E image, and individual cells were segmented and classified as positively or negatively stained. The coordinates of these cells were then projected back to the tumor grading maps that GLASS-AI produced from the original H&E image, assigned to the matching class, and associated with individual tumors.

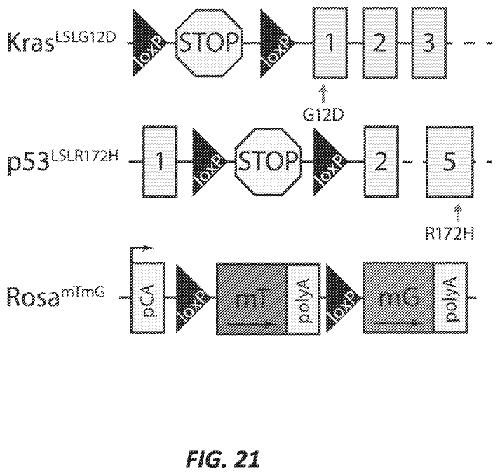

It was found that p-Mek and p-Erk were present in a subset of tumors in both K and TK mice but were largely absent in adjacent normal tissue, in agreement with previous reports on the Kras and Trp53 mutant mouse models¹⁸. To facilitate comparisons to these studies, a Kras^{LSL-G12D/+}; Trp53^{R172H/+} (KP) mouse model depicted in FIG. 21 was analyzed in addition to the Kras^{G12D/+} (K) and TAp73^{Δ/Δ}; Kras^{G12D/+} (TK) mice.

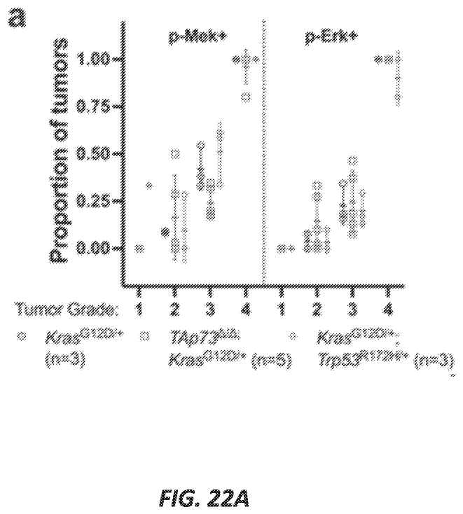

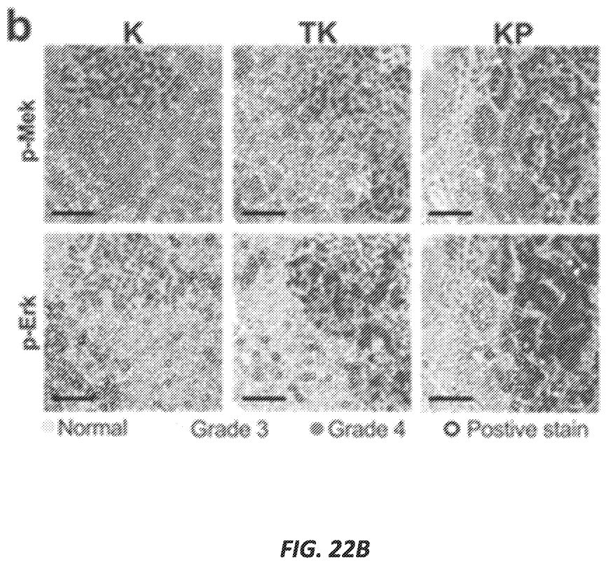

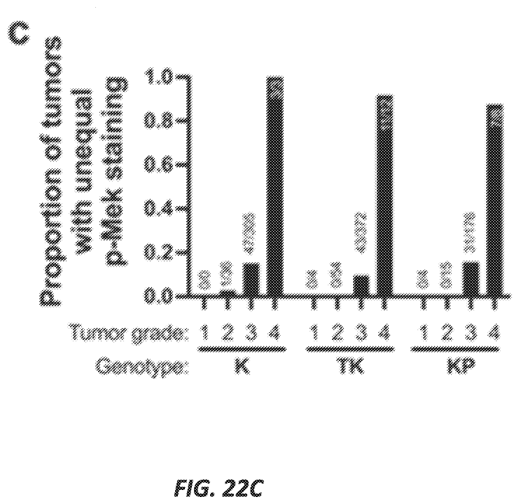

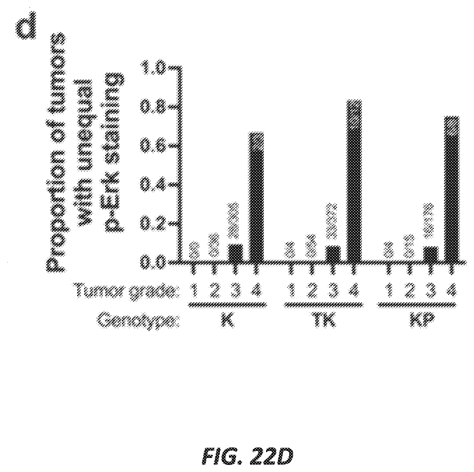

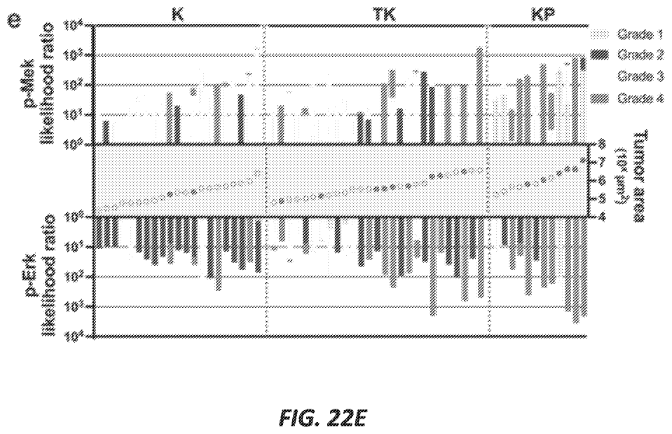

In all three mouse models, both p-Mek and p-Erk positivity increased with tumor grade, and nearly 100% of Grade 4 tumors were positively stained for both markers (FIG. 22A). p-Mek exhibited a broader staining distribution than p-Erk, which primarily appeared in small regions of tumors and occasionally throughout the entire tumor (FIG. 22B). The predominantly focal staining of p-Erk has also been previously reported to occur in high-grade tumors of Kras^{LSL-G12D/+} mice and mice with additional mutations or deletions of Trp53^{18,22}.

The high-resolution tumor grading produced by GLASS-AI facilitated examination of the distribution of p-Mek and p-Erk staining within regions of different grades in a single tumor. Tumors that displayed an uneven distribution of positively stained cells were determined using a likelihood-ratio G-test G=2Σᵢ₌₁⁴ Oᵢ In

$$\frac{{O}_{i}}{{E}_{i}} ,$$

where regions of each grade containing a greater number of positive cells than expected based on the proportion of the tumor area occupied by that grade will produce a positive value while regions with lower than expected proportion of positive cells will produce a negative value. The proportion of tumors with significantly unequal distribution of either p-Mek or p-Erk was very small in Grade 3 or lower tumors. However, most Grade 4 tumors of all three mouse models displayed significantly disproportionate staining for both markers (FIG. 22C-D). The large increase in the proportion of Grade 4 tumors identified by the G-test compared to the lower Grades may indicate that the development of foci of dysregulated Mek/Erk signaling may drive tumor progression.

Based on these observations, it can be hypothesized that the enrichment of p-Mek and p-Erk staining in the high-grade LUAD of the mouse models should occur in the highest grade regions of these tumors. By examining the likelihood ratios of the individual grade regions in each tumor, it was found that K mice displayed the most robust enrichment of p-Mek staining in Grade 3 areas, but such a clear trend was not present in either the TK or KP tumors (FIG. 22E, top). In contrast, TK and KP mice exhibited strong enrichment for p-Erk staining in Grade 4 regions, while K mice showed much weaker and less specific enrichment (FIG. 22E, bottom). The correspondence between overall tumor grade and the most strongly p-Mek/p-Erk enriched regions supports the hypothesis that the loss of Mek/Erk regulation beyond the constitutive activation of the Ras-Raf-Mek-Erk pathway by Ras^{G12D} may drive tumor progression.

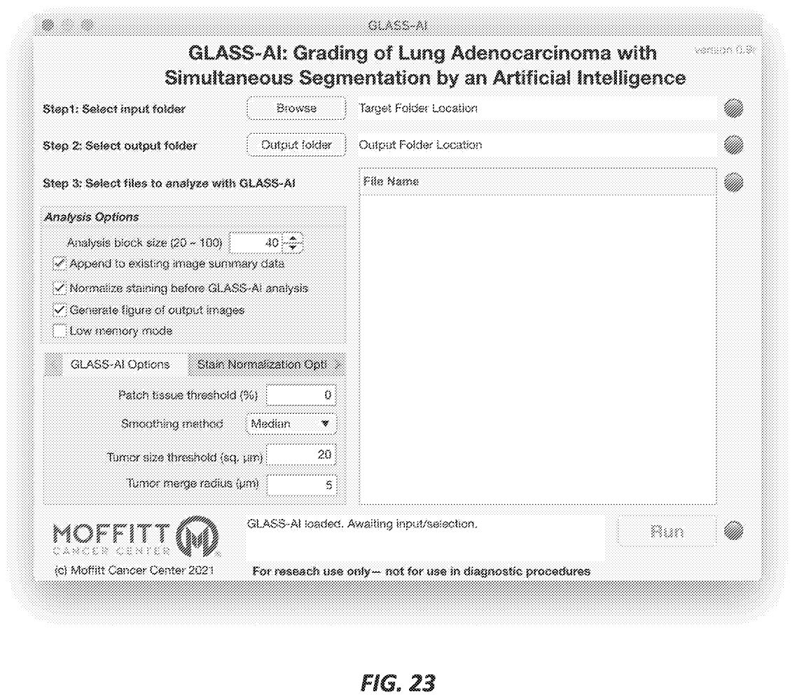

Referring now to FIG. 23, a schematic diagram depicting a user interface in accordance with certain embodiments of the present disclosure is provided. In various embodiments, the example user interface may facilitate interaction with and/or utilization of a GLASS-AI system. The user interface may comprise various features and functionality for accessing, and/or viewing user interface data. The user interface may also comprise messages to the user in the form of banners, headers, notifications, result data (e.g., graphs, chart, images, and the like) and/or the like. As will be recognized, the described elements are provided for illustrative purposes and are not to be construed as limiting the user interface in any way.

Applying machine learning models to digitized WSIs will likely revolutionize how these data are analyzed. Not only can computer vision assist clinicians by providing rapid screening of images, but the higher resolution analysis performed by machine learning models can uncover features that go unnoticed or unreported by human raters. Preclinical studies will also benefit from employing these machine learning models in analysis pipelines by facilitating rapid, reproducible analysis. In accordance with the present disclosure, a purpose-built neural network for grading lung adenocarcinomas in mouse models that provides an unparalleled identification and analysis of tumor grade heterogeneity is provided.

Tumor heterogeneity has been implicated in the progression of many cancer types, including non-small cell lung cancers^{23,24}. Increased intratumor heterogeneity has been linked to decreased overall survival^{23,25,26}, poor response to therapy²⁷, and even increased metastasis²⁸ This heterogeneity is presumed to arise from the clonal evolution of tumor cells within a neoplasm^{29,30}. Typically, tumor heterogeneity is estimated using bulk molecular analyses, such as RNAseq or copy number variation. Previous studies have utilized bulk sample analyses correlated with histomorphological features to predict spatial heterogeneity of molecular markers^{31,32}. However, recent studies have begun using spatially sensitive techniques²⁹ or multi-region sampling³³. Combining these approaches with high-resolution analysis from machine learning pipelines like GLASS-AI may provide an unprecedented understanding of cancer development, progression to metastasis, and treatment response through information derived from spatial genomics, transcriptomics, and proteomics correlated with tumor phenotype.

The recent development of commercially available spatial transcriptomics platforms is a promising step forward in correlating molecular and histological analyses. Some groups have already begun developing machine learning applications utilizing these technologies³⁴. However, these platforms are currently focused on fresh-frozen specimens rather than the FFPE samples typically used for histological analyses in both mouse and human LUAD. Further improvement of these technologies to enable the use of FFPE archival tissues would significantly enhance our understanding of the molecular drivers of tumor progression and heterogeneity and allow the prediction of molecular features from routine histological preparations. This ability to accurately predict molecular markers from simple histology images could be used to flag specimens for further molecular characterization and even provide increased diagnostic and therapeutic choices to clinics without regular access to these molecular techniques. Given the rapid pace of advancement in this field, it seems likely that the first clinical applications of this technique will be realized in the near future.

### Mouse Models and Husbandry

Kras^{LSL-G12D/+}; Rosa^{mTmG/mTmG} (K), Tap73^{fltd/fltd}; Kras^{LSL-G12D/+} (TK), and Kras^{LSL-G12D/+}; Rosa^{mTmG/mTmG}; Trp53^{LSL-R172H/+} (KP), and Kras^{LSL-G12D/+}; Trp53^{fl/fl} mice were generated on a C57BL/6 background. Between 8-10 weeks of age, mice were intratracheally instilled with 7.5×10⁷ PFU of adenovirus containing Cre recombinase under the control of a CMV promoter, as previously described¹⁹. Mice were euthanized 30 weeks after infection, and lungs were collected, fixed overnight in formalin, and embedded in paraffin for further processing. All procedures were approved by the Institutional Animal Care and Use Committee (IACUC) at the University of South Florida.

### Tissue Processing

Formalin-fixed paraffin-embedded (FFPE) lung tissue blocks were sectioned at 4-micron thickness by the Tissue Core at Moffitt Cancer Center. Hematoxylin and eosin (H&E)-stained sections were prepared by the Tissue Core immediately after sectioning. Immunostaining of mouse lung sections was performed overnight at 4° C. in humidified chambers with antibodies against p-Mek1/2 (Ser221) (Cell Signaling Technology Cat #2338, RRID: AB_490903; 1:200) or p-Mapk (Erk1/2) (Thr202/Tyr204) (Cell Signaling Technology Cat #4370, RRID: AB_2315112; 1:400) in 2.5% normal horse serum. The IHC signal was developed using DAB after conjugation with ImmPRESS HRP Horse anti-rabbit IgG PLUS polymer kit (Vector Laboratories Cat #MP-7801). Nuclei were counterstained by immersing the slides in Gill's hematoxylin for 1 minute (Vector Laboratories Cat #H-3401).

### Image Pre-Processing

Whole slide images (WSIs) were generated from H&E and immunostained slides using an Aperio ScanScope AT2 Slide Scanner (Leica) at 20× magnification with a resolution of 0.5 microns/pixel. To improve the consistency of the pipeline on H&E slides with various staining intensities, staining was normalized using Non-Linear Spline Mapping³⁵. WSIs of immunostained sections were co-registered to adjacent H&E-stained sections by a combination of global and local co-registration in MATLAB. The global co-registration was achieved by first applying a rigid co-registration to the whole slide of IHC and aligned to the H&E slide. After the initial rigid alignment, the co-registration was further refined by applying an affine transformation to the IHC slide to ensure tissues were adequately aligned in both slides. The affine co-registration step was lightly applied using only a few iterations to avoid undesired deformation. Local co-registration was then performed by manually aligning tumor regions identified by the pipeline in the H&E image to tumor regions in the IHC slide. WSIs were then divided into 224×224-pixel patches before analysis by GLASS-AI.

### Machine Learning

GLASS-AI was written in MATLAB using the Parallel Processing, Deep Learning, Image Processing, and Computer Vision toolboxes. The standalone applications for Windows and Mac were built using the MATLAB Compiler. The network architecture of GLASS-AI was based on ResNet18²⁰; an 18-layer residual network pre-trained on the ImageNet dataset³⁶. An atrous convolution layer and atrous spatial pyramid pooling layer were added after the final convolutional layer to improve context assimilation in the model. The latent features were then processed with transposed convolution and up-scaling before classification. Finally, after classification, a smoothing layer was added to minimize artifacts from image patch edges. An overview of the network architecture and hyperparameters of GLASS-AI are provided herein.

To construct the training dataset, WSIs from 33 mice (Kras^{G12D/+} n=4, TAp73^{Δ/Δ}; Kras^{G12D/+} n=15, Kras^{G12D/+}; Trp53^{Δ/Δ}, n=14) were manually annotated by three expert raters who segmented and graded each tumor within 11 of the WSIs each. The annotated WSIs were then divided into 224×224-pixel images and corresponding label patches. Patches were then grouped by the annotated class (Normal alveolar, Normal airway, Grade 1 LUAD, Grade 2 LUAD, Grade 3 LUAD, and Grade 4 LUAD) that was most abundant within each patch, however, all the annotations present within these patches was left intact (i.e., a patch that was predominantly Grade 3 could still contain Normal Alveolar and Grade 4 LUAD annotated pixels). 6,000 patches were selected for each class from the respective patch group and split 60/20/20 for training, validation, and testing of the machine learning model after ensuring that patches from an individual slide were only present within a single split. Because each image patch could contain varying amounts of each target class, the area of each of the six target classes in each library was balanced via data augmentation by shifting, skewing, and/or rotating patches in which the underrepresented class was the most abundant class present. Using MATLAB Deep Learning Toolbox and 2 NVIDIA P2000 GPUs, the model was set to train for 20 epochs using adaptive moment estimation on 128-patch minibatches with an initial learning rate of 0.01.

### Statistical Analysis

Data were analyzed using the statistical tests indicated in the figure legends using GraphPad Prism 9 software. p<0.05 was considered statistically significant.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method, comprising:
receiving a digital pathology image of a lung adenocarcinoma (LUAD) tissue sample;
inputting the digital pathology image into an artificial intelligence model; and
grading, using the artificial intelligence model, one or more tumors within the LUAD tissue sample.

2. The method of claim 1, further comprising segmenting, using the artificial intelligence model, the one or more tumors in the digital pathology image.

3. The method of claim 1, wherein the step of grading, using the artificial intelligence model, the one or more tumors comprises assigning each of the one or more tumors to one of a plurality of classes.

4. The method of claim 1, wherein the step of grading, using the artificial intelligence model, the one or more tumors comprises assigning one or more areas within each of the one or more tumors to one of a plurality of classes on a pixel-by-pixel basis or a cell-by-cell basis.

5. The method of claim 4, further comprising, identifying, based at least on the pixel-by-pixel or cell-by-cell assignments, one or more genes of interest or one or more drivers of tumor progression.

6. The method of claim 3, wherein the classes comprises one or more of normal alveolar, normal bronchiolar, Grade 1 LUAD, Grade 2 LUAD, Grade 3 LUAD, Grade 4 LUAD, and Grade 5 LUAD.

7. The method of claim 1, wherein the step of grading, using the artificial intelligence model, the one or more tumors comprises generating graphical display data for a pseudo color map of the one or more tumors.

8. The method of claim 1, further comprising analyzing the one or more tumors.

9. The method of claim 8, wherein the step of analyzing the one or more tumors comprises counting the one or more tumors or characterizing an intratumor heterogeneity of the one or more tumors.

10. The method of claim 1, further comprising performing an immuno-histochemistry (IHC) analysis of the one or more tumors.

11. The method of claim 1, wherein the artificial intelligence model is a machine learning model.

12. The method of claim 11, wherein the machine learning model is a supervised machine learning model.

13. The method of claim 12, wherein the supervised machine learning model is a convolutional neural network (CNN).

14. The method of claim 12, wherein the supervised machine learning model comprises one or more Residual Neural Network (ResNet) layers or components.

15. The method of claim 14, wherein the supervised machine learning model further comprises one or more atrous convolutional layers.

16. The method of claim 14, wherein the supervised machine learning model further comprises one or more transposed convolutional layers.

17. The method of claim 1, wherein the digital pathology image is a hematoxylin & eosin (H&E) stained slide image.

18. The method of claim 1, wherein the LUAD tissue sample is from a mouse.

19. The method of claim 1, wherein the LUAD tissue sample is from a human.

20. A method, comprising:
receiving a first digital pathology image of a first lung adenocarcinoma (LUAD) tissue sample, the first digital pathology image being a hematoxylin & eosin (H&E) stained slide image;
inputting the first digital pathology image into an artificial intelligence model;
grading, using the artificial intelligence model, one or more tumors within the first LUAD tissue sample;
segmenting, using the artificial intelligence model, the one or more tumors in the digital pathology image;
receiving a second digital pathology image comprising a second lung adenocarcinoma (LUAD) tissue sample, the second digital pathology image being an immuno-stained slide image;
identifying and classifying a plurality of positively and negatively stained cells within the second LUAD tissue sample;
co-registering the first and second digital pathology images; and
projecting a plurality of respective coordinates of the positively and negatively stained cells within the second LUAD tissue sample onto the one or more tumors within the first LUAD tissue sample.

21. A method, comprising:
training a machine learning model with a dataset, the dataset comprising a plurality of mouse model digital pathology images, each of the mouse model digital pathology images being of a respective lung adenocarcinoma (LUAD) tissue sample from a mouse;
receiving a digital pathology image of a LUAD tissue sample from a human;
inputting the digital pathology image into the trained machine learning model; and
grading, using the trained machine learning model, one or more tumors within the LUAD tissue sample from the human.
